(19) United States
(12) Patent Application Publication
(10) Pub. No.: US 2022/0296563 A1
(43) Pub. Date: Sep. 22, 2022

(54) ACYLATED ACTIVE AGENTS AND METHODS OF THEIR USE FOR THE TREATMENT OF METABOLIC DISORDERS AND NONALCOHOLIC FATTY LIVER DISEASE

(71) Applicant: Flagship Pioneering Innovations V, Inc., Cambridge, MA (US)

(72) Inventors: John Patrick CASEY, JR., Boston, MA (US); David Arthur BERRY, Newton, MA (US); Timothy F. BRIGGS, Waltham, MA (US); Leonard BUCKBINDER, East Greenwich, RI (US); Mi-Jeong KIM, Boston, MA (US); Anna LIANG, Everett, MA (US)

(21) Appl. No.: 17/831,535

(22) Filed: Jun. 3, 2022

Related U.S. Application Data
(63) Continuation of application No. PCT/US2020/063281, filed on Dec. 4, 2020.
(60) Provisional application No. 63/074,785, filed on Sep. 4, 2020.
(60) Provisional application No. 62/944,282, filed on Dec. 5, 2019.

(51) Int. Cl. A61K 31/353; A61K 31/225; A61K 31/7004; A61P 1/16

(52) U.S. Cl. CPC A61K 31/353; A61K 31/225; A61K 31/7004; A61P 1/16

(57) ABSTRACT

Disclosed herein are acylated active agents (e.g., acylated hydroxybenzoic acid), compositions containing them, unit dosage forms containing them, and methods of their use, e.g., for treating a metabolic disorder or nonalcoholic fatty liver disease or for modulating a metabolic marker or nonalcoholic fatty liver disease marker.

### FIELD OF THE INVENTION

The invention relates to compounds and methods of their medicinal use.

### BACKGROUND

The increase in obesity incidence has reached epidemic proportions in the Western world and more recently also in developing countries. Obesity is associated with significant co-morbidities such as cardiovascular diseases and type II diabetes. While bariatric surgery is a known treatment for obesity, this treatment is costly and risky. Pharmacological intervention is typically less efficacious and is often associated with adverse events.

Nonalcoholic fatty liver disease (NAFLD) is one of the most common forms of chronic liver disease, affecting an estimated 12% to 25% people in the United States. The main characteristic of NAFLD is fat accumulation (steatosis) in the liver. In NAFLD, the fat accumulation is not associated with excessive alcohol consumption.

Nonalcoholic steatohepatitis (NASH) is an advanced form of NAFLD. NASH is marked by liver inflammation, which may progress to scarring and irreversible liver damage. At its most severe, NASH can progress to cirrhosis and liver failure.

There is a need for methods and compositions useful for managing metabolic disorders and/or for treating NAFLD and NASH.

### SUMMARY OF THE INVENTION

The invention provides acylated hydroxybenzoic acids, pharmaceutically acceptable salts thereof, and esters thereof, and pharmaceutical compositions, dietary supplements, and food products that include such acylated hydroxybenzoic acids, pharmaceutically acceptable salts thereof, or esters thereof.

In one aspect, the invention provides a unit dosage form comprising at least 0.5 g of a compound of formula (I):

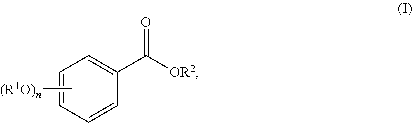

or a pharmaceutically acceptable salt thereof, wherein:

n is 1, 2, 3, 4, or 5;

each R¹ is independently H, alkyl, or acyl; and

R² is H or alkyl;

provided that the compound comprises at least one fatty acid acyl.

In some embodiments, n is 2. In some embodiments, the compound is a compound of formula (IA):

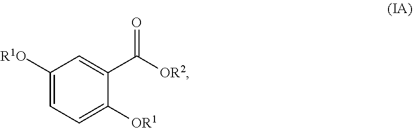

or a pharmaceutically acceptable salt thereof.

In some embodiments, each R¹ is independently acyl. In some embodiments, each R¹ is independently a short chain fatty acid acyl. In some embodiments, the compound is:

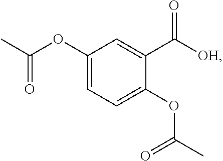 [SMILES: CC(=O)Oc1ccc(OC(C)=O)c(C(=O)O)c1]

or a pharmaceutically acceptable salt thereof.

In some embodiments, the unit dosage form includes at least 1 g (e.g., at least 2 g) of the active agent. In some embodiments, the unit dosage form includes 10 g or less (e.g., 9 g or less, 8 g or less, 7 g or less, 6 g or less, or 5 g or less) of the active agent. In some embodiments, the unit dosage form includes 0.5 to 10 g (e.g., 1 to 10 g, 2 to 10 g, 3 to 10 g, 4 to 10 g, 5 to 10 g, 6 to 10 g, 7 to 10 g, 8 to 10 g, 9 to 10 g, 0.5 to 9 g, 1 to 9 g, 2 to 9 g, 3 to 9 g, 4 to 9 g, 5 to 9 g, 6 to 9 g, 7 to 9 g, 8 to 9 g, 0.5 to 8 g, 1 to 8 g, 2 to 8 g, 3 to 8 g, 4 to 8 g, 5 to 8 g, 6 to 8 g, 7 to 8 g, 0.5 to 7 g, 1 to 7 g, 2 to 7 g, 3 to 7 g, 4 to 7 g, 5 to 7 g, 6 to 7 g, 0.5 to 6 g, 1 to 6 g, 2 to 6 g, 3 to 6 g, 4 to 6 g, 5 to 6 g, 0.5 to 5 g, 1 to 5 g, 2 to 5 g, 3 to 5 g, 4 to 5 g, 0.5 to 4 g, 1 to 4 g, 2 to 4 g, 3 to 4 g, 0.5 to 3 g, 1 to 3 g, 2 to 3 g, 0.5 to 2 g, 1 to 2 g, or 0.5 to 1 g) of the active agent.

In some embodiments, the unit dosage form is a food additive unit dosage form, a pharmaceutical unit dosage form, or a dietary supplement unit dosage form. In some embodiments, the unit dosage form is a food additive unit dosage form that is a serving of a food product. In some embodiments, the unit dosage form is a pharmaceutical unit dosage form. In some embodiments, the unit dosage form is a dietary supplement unit dosage form.

In another aspect, the invention provides a method of modulating a metabolic marker or a nonalcoholic fatty liver disease marker, the method including administering an effective amount of an active agent to a subject in need thereof, where the active agent is an acylated hydroxybenzoic acid, a pharmaceutically acceptable salt thereof, or an ester thereof.

In some embodiments, the method is for modulating a metabolic marker. In some embodiments, the metabolic marker is for an obesity disorder. In some embodiments, the metabolic marker is for type II diabetes, prediabetes, insulin resistance, metabolic syndrome, hypercholesterolemia, or hyperlipidemia.

In some embodiments, the method is for modulating a nonalcoholic fatty liver disease marker.

In another aspect, the invention provides a method of treating a metabolic disorder or nonalcoholic fatty liver disease, the method including administering an effective amount of an active agent to a subject in need thereof, where the active agent is an acylated hydroxybenzoic acid, or a pharmaceutically acceptable salt thereof, or an ester thereof.

In some embodiments, the method is for treating a metabolic disorder. In some embodiments, the metabolic disorder is an obesity disorder. In some embodiments, the metabolic disorder is type II diabetes, prediabetes, insulin resistance, metabolic syndrome, hypercholesterolemia, or hyperlipidemia.

In some embodiments, the method is for treating nonalcoholic fatty liver disease. In some embodiments, the subject suffers from or is diagnosed with nonalcoholic steatohepatitis.

In some embodiments, the method treats or reduces liver fibrosis.

In another aspect, the invention provides a method of improving glucose or insulin tolerance, of reducing cholesterol levels, of reducing blood sugar levels, or of maintaining a healthy body weight in a subject in need thereof, the method including administering to the subject an effective amount of an active agent to a subject in need thereof, where the active agent is an acylated hydroxybenzoic acid, a pharmaceutically acceptable salt thereof, or an ester thereof.

In some embodiments, the subject is suffering from type II diabetes, prediabetes, insulin resistance, metabolic syndrome, or hypercholesterolemia.

In some embodiments, the method is of improving glucose tolerance. In some embodiments, the method is of improving insulin tolerance. In some embodiments, the method is of reducing blood sugar levels (e.g., the blood sugar levels are elevated prior to the administering step). In some embodiments, the method is of reducing cholesterol levels. In some embodiments, the method is of maintaining a healthy body weight. In some embodiments, the subject has a BMI of 25 or greater prior to the administering step. In some embodiments, the subject has a BMI of less than 25 after the administering step.

In some embodiments, the cholesterol levels are total blood cholesterol levels. In some embodiments, the subject has a total blood cholesterol level of 240 mg/dL or greater prior to the administering step. In some embodiments, the subject has a total blood cholesterol level of less than 240 mg/dL (e.g., less than 200 mg/dL) after the administering step. In some embodiments, the cholesterol levels are serum LDL levels. In some embodiments, the subject has a serum LDL level of 160 mg/dL or greater prior to the administering step. In some embodiments, the subject has a serum LDL level of less than 160 mg/dL (e.g., less than 130 mg/dL) after the administering step.

In some embodiments, total fat percentage, cellular adiposity, body mass index, rate of weight gain, abdominal fat quantity, ratio of white to brown fat, level of lipogenesis, or level of fat storage is reduced following the step of administering. In some embodiments, total fat percentage, cellular adiposity, body mass index, abdominal fat quantity, or ratio of white to brown fat is reduced following the step of administering.

In some embodiments, the subject is overweight. In some embodiments, the subject suffers from obesity. In some embodiments, the subject suffers from severe obesity, morbid obesity, or super obesity. In some embodiments, the subject has a body mass index of at least 25 kg/m². In some embodiments, the subject has a body mass index of at least 28 kg/m². In some embodiments, the subject has a body mass index of at least 30 kg/m². In some embodiments, the subject has a body mass index of at least 35 kg/m². In some embodiments, the subject has a body mass index of at least 45 kg/m².

In some embodiments, the level of insulin, GLP-1, or PYY is increased following the administration of the active agent to the subject. In some embodiments, the level of blood sugar or hemoglobin A1c is reduced following the administration of the active agent to the subject. In some embodiments, the glucose tolerance is increased following the administration of the active agent to the subject.

In some embodiments, the method reduces the level of alanine transaminase in the blood of the subject by at least 1% relative to the level of alanine transaminase in the blood of the subject prior to the administering step. In some embodiments, the method reduces the level of aspartate transaminase in the blood of the subject by at least 1% relative to the level of aspartate transaminase in the blood of the subject prior to the administering step. In some embodiments, the method reduces the liver weight of the subject by at least 1% relative to the liver weight of the subject prior to the administering step.

In some embodiments, the subject is a human. In some embodiments, the subject is a cat or dog.

In some embodiments, the method includes orally administering the active agent to the subject.

In some embodiments, following oral administration to the subject, the active agent is cleavable in the gastrointestinal tract of the subject. In some embodiments, upon cleavage, the active agent releases at least one fatty acid.

In some embodiments, the fatty acid is a short chain fatty acid. In some embodiments, the short chain fatty acid is acetic acid, propionic acid, or butyric acid. In some embodiments, the short chain fatty acid is acetic acid.

In some embodiments, the active agent comprises gentisic acid.

In some embodiments, the active agent is a compound of formula (I):

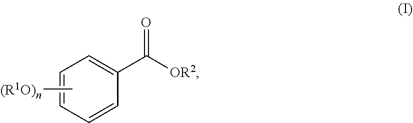

or a pharmaceutically acceptable salt thereof, wherein:

n is 1, 2, 3, 4, or 5;

each R¹ is independently H, alkyl, or acyl; and

R² is H or alkyl;

provided that the compound comprises at least one fatty acid acyl.

In some embodiments, n is 2.

In some embodiments, the compound is a compound of formula (IA):

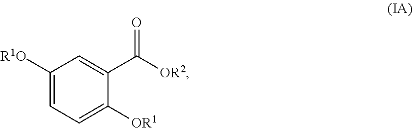

or a pharmaceutically acceptable salt thereof.

In some embodiments, each R¹ is independently acyl.

In some embodiments, each R¹ is independently a short chain fatty acid acyl.

In some embodiments, the compound is:

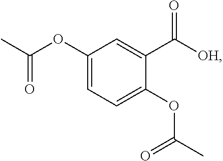 [SMILES: CC(=O)Oc1ccc(OC(C)=O)c(C(=O)O)c1]

or a pharmaceutically acceptable salt thereof.

In some embodiments of any method of the invention, the active agent is administered at a dose of at least 1 g (e.g., at least 2 g) of the active agent. In some embodiments, the active agent is administered at a dose of 10 g or less (e.g., 9 g or less, 8 g or less, 7 g or less, 6 g or less, or 5 g or less) of the active agent. In some embodiments, the active agent is administered at a dose of 0.5 to 10 g (e.g., 1 to 10 g, 2 to 10 g, 3 to 10 g, 4 to 10 g, 5 to 10 g, 6 to 10 g, 7 to 10 g, 8 to 10 g, 9 to 10 g, 0.5 to 9 g, 1 to 9 g, 2 to 9 g, 3 to 9 g, 4 to 9 g, 5 to 9 g, 6 to 9 g, 7 to 9 g, 8 to 9 g, 0.5 to 8 g, 1 to 8 g, 2 to 8 g, 3 to 8 g, 4 to 8 g, 5 to 8 g, 6 to 8 g, 7 to 8 g, 0.5 to 7 g, 1 to 7 g, 2 to 7 g, 3 to 7 g, 4 to 7 g, 5 to 7 g, 6 to 7 g, 0.5 to 6 g, 1 to 6 g, 2 to 6 g, 3 to 6 g, 4 to 6 g, 5 to 6 g, 0.5 to 5 g, 1 to 5 g, 2 to 5 g, 3 to 5 g, 4 to 5 g, 0.5 to 4 g, 1 to 4 g, 2 to 4 g, 3 to 4 g, 0.5 to 3 g, 1 to 3 g, 2 to 3 g, 0.5 to 2 g, 1 to 2 g, or 0.5 to 1 g) of the active agent.

### Definitions

The term “acyl,” as used herein, represents a chemical substituent of formula —C(O)—R, where R is alkyl, alkenyl, aryl, arylalkyl, cycloalkyl, heterocyclyl, heterocyclyl alkyl, heteroaryl, or heteroaryl alkyl; or R combines with —C(O)— to form a fatty acid acyl. An optionally substituted acyl is an acyl that is optionally substituted as described herein for each group R. Non-limiting examples of acyl include fatty acid acyls (e.g., short chain fatty acid acyls, e.g., acetyl).

The term “acylated active agent,” as used herein, represents a compound including two or more agents linked through ester bond(s). Non-limiting examples of acylated active agents include an acylated hydroxybenzoic acid.

The term “acylated hydroxybenzoic acid,” as used herein, represents a compound of formula (I):

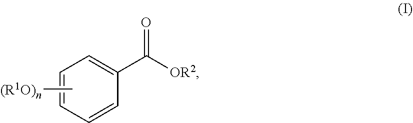

or a pharmaceutically acceptable salt thereof,

where

n is 1, 2, 3, 4, or 5;

each R¹ is independently H, alkyl, or acyl; and

R² is H or alkyl;

provided that the compound comprises at least one acyl (e.g., a fatty acid acyl). Non-limiting examples of acylated hydroxybenzoic acids include gentisic acid, in which one or two phenolic hydroxyls are independently substituted with an acyl.

The term “acyloxy,” as used herein, represents a chemical substituent of formula —OR, where R is acyl. An optionally substituted acyloxy is an acyloxy that is optionally substituted as described herein for acyl.

The term “alcohol oxygen atom,” as used herein, refers to a divalent oxygen atom, where one valency of the alcohol oxygen atom is bonded to a first carbon atom, and another valency is bonded to a second carbon atom, where the first carbon atom is an sp³-hybridized carbon atom, and the second carbon atom is an sp³-hybridized carbon atom or an sp²-hybridized carbon atom of a carbonyl group.

The term “alkanoyl,” as used herein, represents a chemical substituent of formula —C(O)—R, where R is alkyl. An optionally substituted alkanoyl is an alkanoyl that is optionally substituted as described herein for alkyl.

The term “alkoxy,” as used herein, represents a chemical substituent of formula —OR, where R is a C₁₋₆ alkyl group, unless otherwise specified. An optionally substituted alkoxy is an alkoxy group that is optionally substituted as defined herein for alkyl.

The term “alkenyl,” as used herein, represents acyclic monovalent straight or branched chain hydrocarbon groups containing one, two, or three carbon-carbon double bonds. Alkenyl, when unsubstituted, has from 2 to 22 carbons, unless otherwise specified. In certain preferred embodiments, alkenyl, when unsubstituted, has from 2 to 12 carbon atoms (e.g., 1 to 8 carbons). Non-limiting examples of the alkenyl groups include ethenyl, prop-1-enyl, prop-2-enyl, 1-methylethenyl, but-1-enyl, but-2-enyl, but-3-enyl, 1-methylprop-1-enyl, 2-methylprop-1-enyl, and 1-methylprop-2-enyl. Alkenyl groups may be optionally substituted as defined herein for alkyl.

The term “alkyl,” as used herein, refers to an acyclic straight or branched chain saturated hydrocarbon group, which, when unsubstituted, has from 1 to 22 carbons (e.g., 1 to 20 carbons), unless otherwise specified. In certain preferred embodiments, alkyl, when unsubstituted, has from 1 to 12 carbons (e.g., 1 to 8 carbons). Alkyl groups are exemplified by methyl; ethyl; n- and iso-propyl; n-, sec-, iso- and tert-butyl; neopentyl, and the like, and may be optionally substituted, valency permitting, with one, two, three, or, in the case of alkyl groups of two carbons or more, four or more substituents independently selected from the group consisting of: alkoxy; acyloxy; alkylsulfenyl; alkylsulfinyl; alkylsulfonyl; amino; aryl; aryloxy; azido; cycloalkyl; cycloalkoxy; halo; heterocyclyl; heteroaryl; heterocyclylalkyl; heteroarylalkyl; heterocyclyloxy; heteroaryloxy; hydroxy; nitro; thioalkyl; thioalkenyl; thioaryl; thiol; silyl; cyano; oxo (═S); thio (═S); and imino (═NR′), where R′ is H, alkyl, aryl, or heterocyclyl. Each of the substituents may itself be unsubstituted or, valency permitting, substituted with unsubstituted substituent(s) defined herein for each respective group.

The term “alkylsulfenyl,” as used herein, represents a group of formula —S-(alkyl). An optionally substituted alkylsulfenyl is an alkylsulfenyl that is optionally substituted as described herein for alkyl.

The term “alkylsulfinyl,” as used herein, represents a group of formula —S(O)-(alkyl). An optionally substituted alkylsulfinyl is an alkylsulfinyl that is optionally substituted as described herein for alkyl.

The term “alkylsulfonyl,” as used herein, represents a group of formula —S(O)₂-(alkyl). An optionally substituted alkylsulfonyl is an alkylsulfonyl that is optionally substituted as described herein for alkyl.

The term “aryl,” as used herein, represents a mono-, bicyclic, or multicyclic carbocyclic ring system having one or two aromatic rings. Aryl group may include from 6 to 10 carbon atoms. All atoms within an unsubstituted carbocyclic aryl group are carbon atoms. Non-limiting examples of carbocyclic aryl groups include phenyl, naphthyl, 1,2-dihydronaphthyl, 1,2,3,4-tetrahydronaphthyl, fluorenyl, indanyl, indenyl, etc. The aryl group may be unsubstituted or substituted with one, two, three, four, or five substituents independently selected from the group consisting of: alkyl; alkenyl; alkoxy; acyloxy; amino; aryl; aryloxy; azido; cycloalkyl; cycloalkoxy; halo; heterocyclyl; heteroaryl; heterocyclylalkyl; heteroarylalkyl; heterocyclyloxy; heteroaryloxy; hydroxy; nitro; thioalkyl; thioalkenyl; thioaryl; thiol; silyl; and cyano. Each of the substituents may itself be unsubstituted or substituted with unsubstituted substituent(s) defined herein for each respective group.

The term “aryl alkyl,” as used herein, represents an alkyl group substituted with an aryl group. An optionally substituted aryl alkyl is an aryl alkyl, in which aryl and alkyl portions may be optionally substituted as the individual groups as described herein.

The term “aryloxy,” as used herein, represents a group —OR, where R is aryl. Aryloxy may be an optionally substituted aryloxy. An optionally substituted aryloxy is aryloxy that is optionally substituted as described herein for aryl.

The term “carbonyl,” as used herein, refers to a divalent group —C(O)—.

The term “carboxylate,” as used herein, represents group —COON or a pharmaceutically acceptable salt thereof.

The expression “C_{x-y},” as used herein, indicates that the group, the name of which immediately follows the expression, when unsubstituted, contains a total of from x to y carbon atoms. If the group is a composite group (e.g., aryl alkyl), C_{x-y} indicates that the portion, the name of which immediately follows the expression, when unsubstituted, contains a total of from x to y carbon atoms. For example, (C₆₋₁₀-aryl)-C₁₋₆-alkyl is a group, in which the aryl portion, when unsubstituted, contains a total of from 6 to 10 carbon atoms, and the alkyl portion, when unsubstituted, contains a total of from 1 to 6 carbon atoms.

The term “cycloalkyl,” as used herein, refers to a cyclic alkyl group having from three to ten carbons (e.g., a C₃-C₁₀ cycloalkyl), unless otherwise specified. Cycloalkyl groups may be monocyclic or bicyclic. Bicyclic cycloalkyl groups may be of bicyclo[p.q.0]alkyl type, in which each of p and q is, independently, 1, 2, 3, 4, 5, 6, or 7, provided that the sum of p and q is 2, 3, 4, 5, 6, 7, or 8. Alternatively, bicyclic cycloalkyl groups may include bridged cycloalkyl structures, e.g., bicyclo[p.q.r]alkyl, in which r is 1, 2, or 3, each of p and q is, independently, 1, 2, 3, 4, 5, or 6, provided that the sum of p, q, and r is 3, 4, 5, 6, 7, or 8. The cycloalkyl group may be a spirocyclic group, e.g., spiro[p.q]alkyl, in which each of p and q is, independently, 2, 3, 4, 5, 6, or 7, provided that the sum of p and q is 4, 5, 6, 7, 8, or 9. Non-limiting examples of cycloalkyl include cyclopropyl, cyclobutyl, cyclopentyl, cyclohexyl, cycloheptyl, 1-bicyclo[2.2.1]heptyl, 2-bicyclo[2.2.1]heptyl, 5-bicyclo[2.2.1]heptyl, 7-bicyclo[2.2.1]heptyl, and decalinyl. The cycloalkyl group may be unsubstituted or substituted (e.g., optionally substituted cycloalkyl) with one, two, three, four, or five substituents independently selected from the group consisting of: alkyl; alkenyl; alkoxy; acyloxy; alkylsulfenyl; alkylsulfinyl; alkylsulfonyl; amino; aryl; aryloxy; azido; cycloalkyl; cycloalkoxy; halo; heterocyclyl; heteroaryl; heterocyclylalkyl; heteroarylalkyl; heterocyclyloxy; heteroaryloxy; hydroxy; nitro; thioalkyl; thioalkenyl; thioaryl; thiol; silyl; cyano; oxo (═O); thio (═S); imino (═NR′), where R′ is H, alkyl, aryl, or heterocyclyl. Each of the substituents may itself be unsubstituted or substituted with unsubstituted substituent(s) defined herein for each respective group.

The term “cycloalkoxy,” as used herein, represents a group —OR, where R is cycloalkyl. An optionally substituted cycloalkoxy is cycloalkoxy that is optionally substituted as described herein for cycloalkyl.

The term “elevated blood sugar levels,” as used herein, refers to a fasting blood sugar level of a subject (e.g., a subject suffering from type II diabetes, prediabetes, or insulin resistance) that is higher than 130 mg/dL or to the blood sugar level of a subject that is higher than 180 mg/dL (e.g., a subject suffering from type II diabetes, prediabetes, or insulin resistance) two hours after a meal. The term “elevated blood sugar levels” may alternatively refer to a fasting blood sugar level higher than 100 mg/dL for a subject not suffering from type II diabetes, prediabetes, or insulin resistance. The term “elevated blood sugar levels” may alternatively refer to a blood sugar level higher than 140 mg/dL two hours after a meal for a subject not suffering from type II diabetes, prediabetes, or insulin resistance. A fasting blood sugar level of 70 mg/dL to 130 mg/dL is considered a normal fasting blood sugar level for a subject (e.g., a subject suffering from type II diabetes, prediabetes, or insulin resistance). A fasting blood sugar level of 70 mg/dL to 100 mg/dL is considered a normal fasting blood sugar level for a subject not suffering from type II diabetes, prediabetes, or insulin resistance. The blood sugar levels can be measured using methods known in the art.

The term “ester bond,” as used herein, refers to a covalent bond between an alcohol or phenolic oxygen atom and a carbonyl group that is further bonded to a carbon atom.

The term “fatty acid,” as used herein, refers to a short-chain fatty acid, a medium chain fatty acid, a long chain fatty acid, a very long chain fatty acid, or an unsaturated analogue thereof, or a phenyl-substituted analogue thereof. Short chain fatty acids contain from 1 to 6 carbon atoms, medium chain fatty acids contain from 7 to 13 carbon atoms, and a long-chain fatty acids contain from 14 to 22 carbon atoms. A fatty acid may be saturated or unsaturated. An unsaturated fatty acid includes 1, 2, 3, 4, 5, or 6 carbon-carbon double bonds. Preferably, the carbon-carbon double bonds in unsaturated fatty acids have Z stereochemistry.

The term “fatty acid acyl,” as used herein, refers to a fatty acid, in which the hydroxyl group is replaced with a valency.

The term “fatty acid acyloxy,” as used herein, refers to group —OR, where R is a fatty acid acyl.

The term “halogen,” as used herein, represents a halogen selected from bromine, chlorine, iodine, and fluorine.

The term “healthy body weight,” as used herein, refers to a body mass index (BMI) range recognized as a normal weight range. For example, World Health Organization and U.S. Center for Disease Control recognize the BMI range of 18.5 kg/m² to less than 25 kg/m² to be a normal weight range for humans. World Health Organization and U.S. Center for Disease Control recognize the BMI range of 25 kg/m² to less than 30 kg/m² as “overweight” and the BMI range of 30 kg/m² or higher as “obese” for humans. An overweight human is one having the BMI of 25 kg/m² to less than 30 kg/m². An obese human is one having the BMI of 30 kg/m² or higher.

The term “heteroaryl,” as used herein, represents a monocyclic 5-, 6-, 7-, or 8-membered ring system, or a fused or bridging bicyclic, tricyclic, or tetracyclic ring system; the ring system contains one, two, three, or four heteroatoms independently selected from the group consisting of nitrogen, oxygen, and sulfur; and at least one of the rings is an aromatic ring. Non-limiting examples of heteroaryl groups include benzimidazolyl, benzofuryl, benzothiazolyl, benzothienyl, benzoxazolyl, furyl, imidazolyl, indolyl, isoindazolyl, isoquinolinyl, isothiazolyl, isothiazolyl, isoxazolyl, oxadiazolyl, oxazolyl, purinyl, pyrrolyl, pyridinyl, pyrazinyl, pyrimidinyl, qunazolinyl, quinolinyl, thiadiazolyl (e.g., 1,3,4-thiadiazole), thiazolyl, thienyl, triazolyl, tetrazolyl, dihydroindolyl, tetrahydroquinolyl, tetrahydroisoquinolyl, etc. The term bicyclic, tricyclic, and tetracyclic heteroaryls include at least one ring having at least one heteroatom as described above and at least one aromatic ring. For example, a ring having at least one heteroatom may be fused to one, two, or three carbocyclic rings, e.g., an aryl ring, a cyclohexane ring, a cyclohexene ring, a cyclopentane ring, a cyclopentene ring, or another monocyclic heterocyclic ring. Examples of fused heteroaryls include 1,2,3,5,8,8a-hexahydroindolizine; 2,3-dihydrobenzofuran; 2,3-dihydroindole; and 2,3-dihydrobenzothiophene. Heteroaryl may be optionally substituted with one, two, three, four, or five substituents independently selected from the group consisting of: alkyl; alkenyl; alkoxy; acyloxy; aryloxy; alkylsulfenyl; alkylsulfinyl; alkylsulfonyl; amino; arylalkoxy; cycloalkyl; cycloalkoxy; halogen; heterocyclyl; heterocyclyl alkyl; heteroaryl; heteroaryl alkyl; heterocyclyloxy; heteroaryloxy; hydroxyl; nitro; thioalkyl; thioalkenyl; thioaryl; thiol; cyano; ═O; —NR₂, where each R is independently hydrogen, alkyl, acyl, aryl, arylalkyl, cycloalkyl, heterocyclyl, or heteroaryl; —COOR^{A}, where R^{A} is hydrogen, alkyl, aryl, arylalkyl, cycloalkyl, heterocyclyl, or heteroaryl; and —CON(R^{B})₂, where each R^{B} is independently hydrogen, alkyl, aryl, arylalkyl, cycloalkyl, heterocyclyl, or heteroaryl. Each of the substituents may itself be unsubstituted or substituted with unsubstituted substituent(s) defined herein for each respective group.

The term “heteroaryl alkyl,” as used herein, represents an alkyl group substituted with a heteroaryl group. The heteroaryl and alkyl portions of an optionally substituted heteroaryl alkyl are optionally substituted as described for heteroaryl and alkyl, respectively.

The term “heteroaryloxy,” as used herein, refers to a structure —OR, in which R is heteroaryl. Heteroaryloxy can be optionally substituted as defined for heteroaryl.

The term “heterocyclyl,” as used herein, represents a monocyclic, bicyclic, tricyclic, or tetracyclic non-aromatic ring system having fused or bridging 4-, 5-, 6-, 7-, or 8-membered rings, unless otherwise specified, the ring system containing one, two, three, or four heteroatoms independently selected from the group consisting of nitrogen, oxygen, and sulfur. Non-aromatic 5-membered heterocyclyl has zero or one double bonds, non-aromatic 6- and 7-membered heterocyclyl groups have zero to two double bonds, and non-aromatic 8-membered heterocyclyl groups have zero to two double bonds and/or zero or one carbon-carbon triple bond. Heterocyclyl groups have a carbon count of 1 to 16 carbon atoms unless otherwise specified. Certain heterocyclyl groups may have a carbon count up to 9 carbon atoms. Non-aromatic heterocyclyl groups include pyrrolinyl, pyrrolidinyl, pyrazolinyl, pyrazolidinyl, imidazolinyl, imidazolidinyl, piperidinyl, homopiperidinyl, piperazinyl, pyridazinyl, oxazolidinyl, isoxazolidiniyl, morpholinyl, thiomorpholinyl, thiazolidinyl, isothiazolidinyl, thiazolidinyl, tetrahydrofuranyl, dihydrofuranyl, tetrahydrothienyl, dihydrothienyl, pyranyl, dihydropyranyl, dithiazolyl, etc. The term “heterocyclyl” also represents a heterocyclic compound having a bridged multicyclic structure in which one or more carbons and/or heteroatoms bridges two non-adjacent members of a monocyclic ring, e.g., quinuclidine, tropanes, or diaza-bicyclo[2.2.2]octane. The term “heterocyclyl” includes bicyclic, tricyclic, and tetracyclic groups in which any of the above heterocyclic rings is fused to one, two, or three carbocyclic rings, e.g., a cyclohexane ring, a cyclohexene ring, a cyclopentane ring, a cyclopentene ring, or another heterocyclic ring. Examples of fused heterocyclyls include 1,2,3,5,8,8a-hexahydroindolizine; 2,3-dihydrobenzofuran; 2,3-dihydroindole; and 2,3-dihydrobenzothiophene. The heterocyclyl group may be unsubstituted or substituted with one, two, three, four or five substituents independently selected from the group consisting of: alkyl; alkenyl; alkoxy; acyloxy; alkylsulfenyl; alkylsulfinyl; alkylsulfonyl; aryloxy; amino; arylalkoxy; cycloalkyl; cycloalkoxy; halogen; heterocyclyl; heterocyclyl alkyl; heteroaryl; heteroaryl alkyl; heterocyclyloxy; heteroaryloxy; hydroxyl; nitro; thioalkyl; thioalkenyl; thioaryl; thiol; cyano; ═O; ═S; —NR₂, where each R is independently hydrogen, alkyl, acyl, aryl, arylalkyl, cycloalkyl, heterocyclyl, or heteroaryl; —COOR_{A}, where R_{A} is hydrogen, alkyl, aryl, arylalkyl, cycloalkyl, heterocyclyl, or heteroaryl; and —CON(R^{B})₂, where each R^{B} is independently hydrogen, alkyl, aryl, arylalkyl, cycloalkyl, heterocyclyl, or heteroaryl.

The term “heterocyclyl alkyl,” as used herein, represents an alkyl group substituted with a heterocyclyl group. The heterocyclyl and alkyl portions of an optionally substituted heterocyclyl alkyl are optionally substituted as described for heterocyclyl and alkyl, respectively.

The term “heterocyclyloxy,” as used herein, refers to a structure —OR, in which R is heterocyclyl. Heterocyclyloxy can be optionally substituted as described for heterocyclyl.

The term “hydroxybenzoic acid,” as used herein, represents a compound of the following structure:

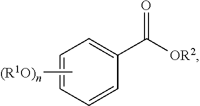

or a pharmaceutically acceptable salt thereof,

where

n is 1, 2, 3, 4, or 5;

each R¹ is independently H or alkyl; and

R² is H or alkyl.

Non-limiting examples of hydroxybenzoic acids include gentisic acid. When R² is an alkyl, the compound is an “ester.”

The terms “hydroxyl” and “hydroxy,” as used interchangeably herein, represent -OH. A hydroxyl substituted with an acyl is an acyloxy. A protected hydroxyl is a hydroxyl, in which the hydrogen atom is replaced with an O-protecting group.

The term “metabolic marker,” as used herein, refers to an observable indicative of the presence, absence, or risk of a metabolic disorder. The level of a metabolic marker may directly or inversely correlate with an obesity state. Non-limiting examples of the metabolic markers are a total fat percentage, cellular adiposity, rate of weight gain, abdominal fat quantity, subcutaneous fat quantity, inguinal fat quantity, epididymal fat quantity, ratio of white to brown fat, cholesterol [e.g., high density lipoprotein (HDL) or low density lipoprotein (LDL)] level, and level of triglycerides. In some embodiments, the metabolic marker is a total fat percentage, cellular adiposity, rate of weight gain, abdominal fat quantity, ratio of white to brown fat, cholesterol [e.g., high density lipoprotein (HDL) or low density lipoprotein (LDL)] level, and level of triglycerides. Total fat percentage can be assessed using body mass index. Abdominal fat can be assessed by measuring waist circumference. Ratio or white fat to brown fat can be assessed by measuring the miRNA-92a level, for example, using techniques and methods described in Chen et al., Nat. Commun., 7:11420; ¹⁸F-fludeoxyglucose positron emission tomography/computed tomography, for example, using techniques and methods described in Gerngroß et al., J. Nucl. Med., 58:1104-1110, 2017; magnetic resonance imaging, for example, using techniques and methods described in Chen et al., J. Nucl. Med., 54:1584-1587, 2013.

The term “modulating,” as used herein, refers to an observable change in the level of a marker in a subject, as measured using techniques and methods known in the art for such a measurement. Modulating the marker level in a subject may result in a change of at least 1% relative to prior to administration (e.g., at least 5%, 10%, 15%, 20%, 25%, 30%, 35%, 40%, 45%, 50%, 55%, 60%, 65%, 70%, 75%, 80%, 85%, 90%, 95% or at least 98% or more relative to prior to administration; e.g., up to 100% relative to prior to administration). In some embodiments, modulating is increasing the level of a marker in a subject. Increasing the marker level in a subject may result in an increase of at least 1% relative to prior to administration (e.g., at least 5%, 10%, 15%, 20%, 25%, 30%, 35%, 40%, 45%, 50%, 55%, 60%, 65%, 70%, 75%, 80%, 85%, 90%, 95% or at least 98% or more relative to prior to administration; e.g., up to 100% relative to prior to administration). In other embodiments, modulating is decreasing the level of a marker in a subject. Decreasing the marker level in a subject may result in a decrease of at least 1% relative to prior to administration (e.g., at least 5%, 10%, 15%, 20%, 25%, 30%, 35%, 40%, 45%, 50%, 55%, 60%, 65%, 70%, 75%, 80%, 85%, 90%, 95% or at least 98% or more relative to prior to administration; e.g., up to 100% relative to prior to administration). In embodiments in which a parameter is increased or decreased (or reduced) in a subject following a step of administering a composition described herein, the increase or decrease may take place and/or be detectable within a range of time following the administration (e.g., within six hours, 24 hours, 3 days, a week or longer), and may take place and/or be detectable after one or more administrations (e.g., after 2, 3, 4, 5, 6, 7, 8, 9, 10 or more administrations, e.g., as part of a dosing regimen for the subject).

The term “nonalcoholic fatty disease marker,” as used herein, represents an observable indicative of the presence or absence of a nonalcoholic fatty disease (e.g., nonalcoholic steatohepatitis). The level of a nonalcoholic disease marker may directly or inversely correlate with a nonalcoholic disease state. Non-limiting examples of the nonalcoholic disease markers are the alanine transaminase level (ALT), aspartate transaminase level (AST), γ-glutamyltransferase level, liver weight, and fibrotic markers. The alanine transaminase level, aspartate transaminase level, γ-glutamyltransferase level, and fibrotic markers can be measured in a blood sample from a subject using methods known in the art. Nonalcoholic fatty disease markers can be assessed using non-invasive tests, imaging methods, and biopsy. Liver fibrosis can be assessed invasively via liver biopsy or, alternatively, through non-invasive methods, e.g., composite scores/algorithms of serum markers (Fibrotest, Hepatscore, Fibrometet FIB-4 score, NAFLDD fibrosis score), or imagining techniques including transient elastography, magnetic resonance elastography, acoustic radiation force impulses, and sonography (Almpanis, Z., Annals of Gastroenterology, 29:1-9, 2016). BAAT is an overall clinical score that can be used to identify subjects who would benefit from a liver biopsy for the assessment of a subject for nonalcoholic fatty liver disease (e.g., nonalcoholic steatohepatitis). BAAT combines body mass index, age, ALT, and serum triglycerides. In addition, acoustic radiation force impulse can be used to measure liver stiffness, what correlates with fibrosis scoring. Magnetic Resonance Imaging (MRI) is also used to identify hepatic density and hepatic fat fraction; liver stiffness can be measured by MR elastography (Neuman et al., J. Pharm. Pharm. 19:8-24, 2016).

The term “oxo,” as used herein, represents a divalent oxygen atom (e.g., the structure of oxo may be shown as ═O).

The term “pharmaceutical composition,” as used herein, represents a composition containing a compound described herein, formulated with a pharmaceutically acceptable excipient, and manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other formulation described herein.

The term “pharmaceutically acceptable salt,” as use herein, represents those salts which are, within the scope of sound medical judgment, suitable for use in contact with the tissues of humans and animals without undue toxicity, irritation, allergic response and the like and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P.H. Stahl and C. G. Wermuth), Wiley-VCH, 2008. The salts can be prepared in situ during the final isolation and purification of the compounds described herein or separately by reacting the free base group with a suitable organic acid. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

The term “phenolic oxygen atom,” as used herein, refers to a divalent oxygen atom within the structure of a compound, where one valency of the phenolic oxygen atom is bonded to a first carbon atom, and another valency is bonded to a second carbon atom, where the first carbon atom is an sp²-hybridized carbon atom within a benzene ring, and the second carbon atom is an sp₃-hybridized carbon atom or an sp²-hybridized carbon atom.

The term “protecting group,” as used herein, represents a group intended to protect a hydroxy, an amino, or a carbonyl from participating in one or more undesirable reactions during chemical synthesis. The term “O-protecting group,” as used herein, represents a group intended to protect a hydroxy or carbonyl group from participating in one or more undesirable reactions during chemical synthesis. The term “N-protecting group,” as used herein, represents a group intended to protect a nitrogen containing (e.g., an amino or hydrazine) group from participating in one or more undesirable reactions during chemical synthesis. Commonly used O- and N-protecting groups are disclosed in Greene, “Protective Groups in Organic Synthesis,” 3^{rd} Edition (John Wiley & Sons, New York, 1999), which is incorporated herein by reference. Exemplary O- and N-protecting groups include alkanoyl, aryloyl, or carbamyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, α-chlorobutyryl, benzoyl, 4-chlorobenzoyl, 4-bromobenzoyl, t-butyldimethylsilyl, tri-iso-propylsilyloxymethyl, 4,4′-dimethoxytrityl, isobutyryl, phenoxyacetyl, 4-isopropylpehenoxyacetyl, dimethylformamidino, and 4-nitrobenzoyl.

Exemplary O-protecting groups for protecting carbonyl containing groups include, but are not limited to: acetals, acylals, 1,3-dithianes, 1,3-dioxanes, 1,3-dioxolanes, and 1,3-dithiolanes.

Other O-protecting groups include, but are not limited to: substituted alkyl, aryl, and aryl-alkyl ethers (e.g., trityl; methylthiomethyl; methoxymethyl; benzyloxymethyl; siloxymethyl; 2,2,2,-trichloroethoxymethyl; tetrahydropyranyl; tetrahydrofuranyl; ethoxyethyl; 1-[2-(trimethylsilyl)ethoxy]ethyl; 2-trimethylsilylethyl; t-butyl ether; p-chlorophenyl, p-methoxyphenyl, p-nitrophenyl, benzyl, p-methoxybenzyl, and nitrobenzyl); silyl ethers (e.g., trimethylsilyl; triethylsilyl; triisopropylsilyl; dimethylisopropylsilyl; t-butyldimethylsilyl; t-butyldiphenylsilyl; tribenzylsilyl; triphenylsilyl; and diphenymethylsilyl); carbonates (e.g., methyl, methoxymethyl, 9-fluorenylmethyl; ethyl; 2,2,2-trichloroethyl; 2-(trimethylsilyl)ethyl; vinyl, allyl, nitrophenyl; benzyl; methoxybenzyl; 3,4-dimethoxybenzyl; and nitrobenzyl).

Other N-protecting groups include, but are not limited to, chiral auxiliaries such as protected or unprotected D, L or D, L-amino acids such as alanine, leucine, phenylalanine, and the like; sulfonyl-containing groups such as benzenesulfonyl, p-toluenesulfonyl, and the like; carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxpenzyl oxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxy carbonyl, t-butyloxycarbonyl, diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxy carbonyl, fluorenyl-9-methoxycarbonyl, cyclopentyloxycarbonyl, adamantyloxycarbonyl, cyclohexyloxycarbonyl, phenylthiocarbonyl, and the like, aryl-alkyl groups such as benzyl, triphenylmethyl, benzyloxymethyl, and the like and silyl groups such as trimethylsilyl, and the like. Useful N-protecting groups are formyl, acetyl, benzoyl, pivaloyl, t-butylacetyl, alanyl, phenylsulfonyl, benzyl, t-butyloxycarbonyl (Boc), and benzyloxycarbonyl (Cbz).

The term “subject,” as used herein, represents a human or non-human animal (e.g., a mammal) that is suffering from, or is at risk of, disease, disorder, or condition, as determined by a qualified professional (e.g., a doctor or a nurse practitioner) with or without known in the art laboratory test(s) of sample(s) from the subject. Non-limiting examples of diseases, disorders, and conditions include metabolic disorders, as described herein.

The term “thioalkenyl,” as used herein, represents a group —SR, where R is alkenyl. An optionally substituted thioalkenyl is thioalkenyl that is optionally substituted as described herein for alkenyl.

The term “thioalkyl,” as used herein, represents a group —SR, where R is alkyl. An optionally substituted thioalkyl is thioalkyl that is optionally substituted as described herein for alkyl.

The term “thioaryl,” as used herein, represents a group —SR, where R is aryl. An optionally substituted thioaryl is thioaryl that is optionally substituted as described herein for aryl.

“Treatment” and “treating,” as used herein, refer to the medical management of a subject with the intent to improve, ameliorate, stabilize, prevent or cure a disease, disorder, or condition. This term includes active treatment (treatment directed to improve the disease, disorder, or condition); causal treatment (treatment directed to the cause of the associated disease, disorder, or condition); palliative treatment (treatment designed for the relief of symptoms of the disease, disorder, or condition); preventative treatment (treatment directed to minimizing or partially or completely inhibiting the development of the associated disease, disorder, or condition); and supportive treatment (treatment employed to supplement another therapy).

The compounds described herein, unless otherwise noted, encompass isotopically enriched compounds (e.g., deuterated compounds), tautomers, and all stereoisomers and conformers (e.g., enantiomers, diastereomers, E/Z isomers, atropisomers, etc.), as well as racemates thereof and mixtures of different proportions of enantiomers or diastereomers, or mixtures of any of the foregoing forms as well as salts (e.g., pharmaceutically acceptable salts).

Other features and advantages of the invention will be apparent from the Drawings, Detailed Description, and the Claims.

### BRIEF DESCRIPTION OF THE DRAWINGS

FIGS. 1A and 1B are charts showing average body weights of C57BL/6 mice over time. The mice were divided into 9 cohorts and fed either a low-fat or a high-fat diet. The mice in the LFD cohort were fed a low-fat diet, and the mice in the remaining cohorts were fed a high-fat diet only (HFD) or a high-fat diet with an additional component as indicated in the legend. As shown in FIG. 1B, mice fed either a low-fat diet or a high-fat diet supplemented with compound 3 exhibited a statistically significant reduction in the body weight relative to the high-fat diet control group (p<0.05) at the end of the study (day 84). Mice fed a high-fat diet and treated with semaglutide also exhibited a statistically significant reduction in the body weight relative to the high-fat diet control group (p<0.05) at the end of the study (day 84). In FIG. 1B, * indicates p<0.05 vs. HFD at day 10-day 66 (n=9-12); ** indicates p<0.05 vs. HFD at day 10-day 84 (n=9-12).

FIGS. 1C and 1D are charts showing food consumption by the same C57BL/6 mice cohorts over time. The chart in FIG. 1C terminates at day 80 of the study. The chart in FIG. 1D provides the data for the entire study (84 days).

FIGS. 1E and 1F are charts showing whole blood glucose levels as measured in the same C57BL/6 mice cohorts over time. The chart in FIG. 1E terminates at day 80 of the study. The chart in FIG. 1F provides the data for the entire study (84 days).

FIG. 2A is a chart showing glucose levels post-glucose challenge in the glucose tolerance test for C57BL/6 mice at day 80 of the study.

FIG. 2B is a chart showing glucose area under the curve (AUC) measured in the glucose tolerance test for C57BL/6 mice. An asterisk indicates a statistically significant (p<0.05) reduction of the metric relative to the HFD cohort.

FIG. 3A is a chart showing glucose levels post-insulin challenge in the insulin tolerance test for C57BL/6 mice at day 72 of the study.

FIG. 3B is a chart showing glucose area under the curve (AUC) measured in the insulin tolerance test for C57BL/6 mice. An asterisk indicates a statistically significant (p<0.05) reduction of the metric relative to the HFD cohort.

FIG. 4A is a chart showing fasted glucose levels in C57BL/6 mice at day 58 of the study. An asterisk indicates a statistically significant (p<0.05) reduction of the metric relative to the HFD cohort.

FIG. 4B is a chart showing fasted cholesterol levels in C57BL/6 mice at day 58 of the study. An asterisk indicates a statistically significant (p<0.05) reduction of the metric relative to the HFD cohort.

FIG. 4C is a chart showing fasted high-density lipoprotein (HDL) levels in C57BL/6 mice at day 58 of the study. An asterisk indicates a statistically significant (p<0.05) reduction of the metric relative to the HFD cohort.

FIG. 4D is a chart showing fasted low-density lipoprotein (LDL) levels in C57BL/6 mice at day 58 of the study. An asterisk indicates a statistically significant (p<0.05) reduction of the metric relative to the HFD cohort.

FIG. 4E is a chart showing fasted triglyceride levels in C57BL/6 mice at day 58 of the study. An asterisk indicates a statistically significant (p<0.05) reduction of the metric relative to the HFD cohort.

FIG. 5A is a bar chart showing the serum ALT levels in C57BL/6 mice at the end of the study.

FIG. 5B is a bar chart showing the serum AST levels in C57BL/6 mice at the end of the study.

FIG. 6A is a bar chart showing the serum total, non-fasted cholesterol levels in C57BL/6 mice at the end of the study.

FIG. 6B is a bar chart showing the serum total, non-fasted triglycerides levels in C57BL/6 mice at the end of the study.

FIG. 6C is a bar chart showing the serum total, non-fasted HDL levels in C57BL/6 mice at the end of the study.

FIG. 6D is a bar chart showing the serum total, non-fasted LDL levels in C57BL/6 mice at the end of the study.

FIG. 7A is a bar chart showing the liver triglyceride levels in C57BL/6 mice at the end of the study.

FIG. 7B is a bar chart showing the liver cholesterol levels in C57BL/6 mice at the end of the study.

FIG. 7C is a bar chart showing the liver weights for C57BL/6 mice at the end of the study.

FIG. 8A is a bar chart showing the subcutaneous fat pad weights for C57BL/6 mice at the end of the study.

FIG. 8B is a bar chart showing the epididymal fat pad weights for C57BL/6 mice at the end of the study.

### DETAILED DESCRIPTION

The invention provides acylated active agents (e.g., an acylated hydroxybenzoic acid, pharmaceutically acceptable salts thereof, or esters thereof), compositions containing them (e.g., as unit dosage forms), and methods for modulating a metabolic marker in a subject or of treating a metabolic disorder in a subject. Without wishing to be bound by theory, the acylated active agents of the invention are believed to act in concert with, or in lieu of, the microbiota of a subject.

As described herein, the compounds of the invention were unexpectedly observed to exhibit a superior activity in vivo for modulating a metabolic marker or for treating a metabolic disorder (e.g., obesity, type II diabetes, prediabetes, insulin resistance, metabolic syndrome, hypercholesterolemia, atherosclerosis or hyperlipidemia). It has been surprisingly found that administration of an acylated hydroxybenzoic acid (e.g., diacetyl gentisic acid) to a subject can induce weight loss, reduce cholesterol levels, reduce blood sugar levels, and improve glucose and insulin tolerance, even if the subject is fed a high-fat diet. Surprisingly, administration of an acylated hydroxybenzoic acid (e.g., diacetyl gentisic acid) was found to produce superior activity relative to the administration of certain other acylated active agents and peptidic GLP-1 mimics (e.g., semaglutide).

The components of the acylated active agents (e.g., a short chain fatty acid acyl (e.g., acetyl) in combination with a hydroxybenzoic acid, e.g., gentisic acid) may act synergistically to modulate a metabolic marker, e.g., upon hydrolysis in the GI tract of the subject receiving the acylated active agent. The components of the acylated active agent (e.g., a short chain fatty acid acyl (e.g., acetyl) in combination with a hydroxybenzoic acid, e.g., gentisic acid) may act synergistically to treat a metabolic disorder, e.g., upon hydrolysis in the GI tract of the subject receiving the acylated hydroxybenzoic acid (e.g., diacetyl gentisic acid).

Advantageously, acylated active agents disclosed herein may have superior organoleptic properties (e.g., palatability). This provides an important advantage as the individual components (e.g., a short chain fatty acid acyl (e.g., acetyl) in combination with a hydroxybenzoic acid, e.g., gentisic acid) may exhibit less desirable organoleptic properties (e.g., palatability). Improved organoleptic properties facilitate oral administration and are particularly advantageous for delivery of high unit dosages (e.g., unit dosages of 0.5 g or higher).

### Acylated Active Agents

An acylated active agent disclosed herein may be an acylated hydroxybenzoic acid, a pharmaceutically acceptable salt thereof, or an ester thereof.

An acylated hydroxybenzoic acid may be, e.g., a compound of formula (I):

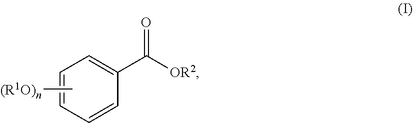

or a pharmaceutically acceptable salt thereof,

where

n is 1, 2, 3, 4, or 5;

each R¹ is independently H, alkyl, or acyl; and

R² is H or alkyl;

provided that the compound comprises at least one acyl (e.g., a fatty acid acyl).

An acylated hydroxybenzoic acid may be, e.g., a compound of formula (IA):

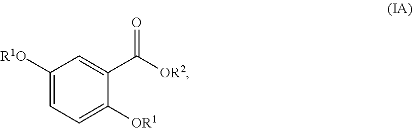

or a pharmaceutically acceptable salt thereof, where each R¹ and R² are independently as described herein for acylated hydroxybenzoic acids.

Non-limiting examples of acylated hydroxybenzoic acids include gentisic acid, in which one or two phenolic hydroxyls are independently substituted with an acyl. For example, an acylated hydroxybenzoic acid may be, e.g.,

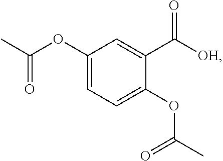 [SMILES: CC(=O)Oc1ccc(OC(C)=O)c(C(=O)O)c1]

or a pharmaceutically acceptable salt thereof.

Pharmaceutical compositions including any of the acylated hydroxybenzoic acids are also included in the invention. Dietary supplements including any of the acylated hydroxybenzoic acids are also included in the invention. Food products including any one of the acylated hydroxybenzoic acids are also included in the invention.

### Methods

Acylated active agents described herein may be used to treat a metabolic disorder or nonalcoholic fatty liver disease in a subject in need thereof. Additionally or alternatively, acylated active agents described herein may be used to modulate a metabolic marker or nonalcoholic fatty liver disease marker in a subject in need thereof. Additionally or alternatively, acylated active agents described herein (e.g., acylated hydroxybenzoic acid, or a pharmaceutically acceptable salt thereof, or an ester thereof, e.g., compound 3) may be used to improve glucose or insulin tolerance, reduce cholesterol levels (preferably, LDL levels), or reduce blood sugar levels in a subject in need thereof. Advantageously, reduction of cholesterol levels (preferably, LDL levels) may reduce the incidence of coronary heart disease among subjects administered the acylated active agent (e.g., acylated hydroxybenzoic acid, or a pharmaceutically acceptable salt thereof, or an ester thereof, e.g., compound 3), e.g., relative to subjects that are not administered the acylated active agent. The relationship between cholesterol levels (e.g., LDL levels) and the incidence of coronary heart disease has been well recognized in the art (e.g., 21 C.F.R. §101.75). Additionally or alternatively, acylated active agents described herein (e.g., acylated hydroxybenzoic acid, or a pharmaceutically acceptable salt thereof, or an ester thereof, e.g., compound 3) may be used to maintain a healthy weight in a subject. Typically, a healthy weight corresponds to a body mass index of less than 25.

Western diets—high in fats and refined carbohydrates—are associated with weight gain leading to obesity and risk for metabolic syndrome, type II diabetes, prediabetes, insulin resistance, hypercholesterolemia, and hyperlipidemia. Consumption of these diets may lead to accumulation of fat in the adipose tissue and liver. This may result in a change in the gut microbiome and elevation of the markers associated with metabolic disorders. In susceptible individuals, these dietary driven changes can lead to outright diabetes. Type II diabetes can cause cardiovascular and ophthalmic disease which can result in blindness, peripheral vascular insufficiency, cardiac disease and premature death. The dietary changes also correlate with changes in the gut microbiome termed dysbiosis. Correcting gut dysbiosis can lead to weight loss and improved glucose tolerance which, longer term, might be expected to abrogate many of the deleterious effects of an unhealthy diet. Metabolic products of the human gut microbiome, such as short chain fatty acids (SFCAs), may produce favorable metabolic effects upon the human host. In some cases, these molecules may work by binding to short chain fatty acid receptors. In other cases, the benefit may be produced via mechanisms such as peroxisome proliferator-activator receptor gamma (PPAR-gamma) or inhibition of histone deacetylase (HDAC).

A method of treating a metabolic disorder in a subject in need thereof may include administering an acylated active agent (e.g., a pharmaceutical or nutraceutical composition containing an acylated active agent) to the subject in need thereof. In some embodiments, the components of the acylated active agent (e.g., a short chain fatty acid acyl (e.g., acetyl) in combination with a hydroxybenzoic acid, e.g., gentisic acid) may act synergistically to treat a metabolic disorder in a subject in need thereof.

Non-limiting examples of metabolic disorders include obesity, metabolic syndrome, type II diabetes, prediabetes, insulin resistance, hypercholesterolemia, atherosclerosis and hyperlipidemia.

A method of modulating a metabolic marker in a subject in need thereof may include administering an acylated active agent (e.g., a pharmaceutical or nutraceutical composition containing an acylated active agent) to the subject. In some embodiments, the components of the acylated active agent (e.g., a short chain fatty acid acyl (e.g., acetyl) in combination with a hydroxybenzoic acid, e.g., gentisic acid) may act synergistically to modulate a metabolic marker in a subject in need thereof.

A method of improving glucose or insulin tolerance, of reducing cholesterol levels, or of reducing blood sugar levels in a subject in need thereof may include administering an acylated active agent (e.g., a pharmaceutical or nutraceutical composition containing an acylated active agent) to the subject. In some embodiments, the components of the acylated active agent (e.g., a short chain fatty acid acyl (e.g., acetyl) in combination with a hydroxybenzoic acid, e.g., gentisic acid) may act synergistically to modulate a metabolic marker in a subject in need thereof. A method of maintaining a healthy body weight in a subject (e.g., a subject in need thereof) may include administering an acylated active agent (e.g., a pharmaceutical or nutraceutical composition containing an acylated active agent) to the subject. In some embodiments, the components of the acylated active agent (e.g., a short chain fatty acid acyl (e.g., acetyl) in combination with a hydroxybenzoic acid, e.g., gentisic acid) may act synergistically to modulate a metabolic marker in a subject in need thereof. The subject may have a BMI of greater than 25 prior to the administering step.

Non-limiting examples of the metabolic markers include markers for obesity, type II diabetes, prediabetes, insulin resistance, metabolic syndrome, hypercholesterolemia, and hyperlipidemia. Obesity markers include, for example, total fat percentage, cellular adiposity, body mass index, rate of weight gain, abdominal fat quantity, subcutaneous fat quantity, inguinal fat quantity, epididymal fat quantity, ratio of white to brown fat, level of lipogenesis, and level of fat storage. Upon administration to a subject in need thereof, an acylated active agent described herein may reduce the total fat percentage, cellular adiposity, body mass index, rate of weight gain, abdominal fat quantity, ratio of white to brown fat, level of lipogenesis, or level of fat storage. Markers for type II diabetes, prediabetes, insulin resistance, metabolic syndrome, hypercholesterolemia, and hyperlipidemia include, for example, an insulin level, GLP-1 level, PYY level, blood sugar level, hemoglobin A1c level, glucose tolerance level, cholesterol (e.g., HDL or LDL) level, and blood triglycerides level. Upon administration to a subject in need thereof, an acylated active agent described herein may increase the insulin level, GLP-1 level, or PYY level. Additionally or alternatively, upon administration to a subject in need thereof, an acylated active agent described herein may reduce the blood sugar level or hemoglobin A1c level. Additionally or alternatively, upon administration to a subject in need thereof, an acylated active agent described herein may increase the glucose tolerance of the subject. Additionally or alternatively, upon administration to a subject in need thereof, an acylated active agent described herein may reduce the blood cholesterol (e.g., LDL) level. Additionally or alternatively, upon administration to a subject in need thereof, an acylated active agent described herein may reduce the blood triglycerides level. In some embodiments, the components of the acylated active agent (e.g., a short chain fatty acid acyl (e.g., acetyl) in combination a hydroxybenzoic acid, e.g., gentisic acid) may act synergistically to modulate a metabolic marker, e.g., upon hydrolysis in the GI tract of the subject receiving the acylated active agent.

In some embodiments, the method maintains the subject within a healthy weight range. In some embodiments, when the subject is overweight or obese, the method reduces the subject's weight, e.g., to a healthy weight range.

In some embodiments, the method reduces the total fat percentage of the subject by at least 1% (e.g., at least 5%, 10%, 15%, 20%, 25%, 30%, 35%, 40%, 45%, 50%, 55%, 60%, 65%, 70%, 75%, 80%, 85%, or 90%, e.g., up to 99%) relative to the total fat percentage of the subject prior to the administering step. In some embodiments, the method reduces the cellular adiposity of the subject by at least 1% (e.g., at least 5′)/0, 10%, 15%, 20%, 25%, 30%, 35%, 40%, 45%, 50%, 55%, 60%, 65%, 70%, 75%, 80%, 85%, or 90%, e.g., up to 99%) relative to the cellular adiposity of the subject prior to the administering step. In some embodiments, the method reduces the body mass index of the subject by at least 1% (e.g., at least 5%, 10%, 15%, 20%, 25%, 30%, 35%, 40%, 45%, or 50%; e.g., up to 60%, 70%, or 80%) relative to the body mass index of the subject prior to the administering step. In some embodiments, the method reduces the rate of weight gain of the subject by at least 1% (e.g., at least 5%, 10%, 15%, 20%, 25%, 30%, 35%, 40%, 45%, 50%, 55%, 60%, 65%, 70%, 75%, 80%, 85%, 90%, 95% or at least 98% or more; e.g., up to 99% or 100%) relative to the rate of weight gain of the subject prior to the administering step. In some embodiments, the method reduces the ratio of white to brown fat in the subject by at least 1% (e.g., at least 5%, 10%, 15%, 20%, 25%, 30%, 35%, 40%, 45%, 50%, 55%, 60%, 65%, 70%, 75%, 80%, 85%, or 90%, e.g., up to 99%) relative to the ratio of white to brown fat in the subject prior to the administering step. In some embodiments, the method reduces the level of lipogenesis in the subject by at least 1% (e.g., at least 5%, 10%, 15%, 20%, 25%, 30%, 35%, 40%, 45%, 50%, 55%, 60%, 65%, 70%, 75%, 80%, 85%, 90%, 95% or at least 98% or more; e.g., up to 99% or 100%) relative to the level of lipogenesis in the subject prior to the administering step. In some embodiments, the method reduces the level of fat storage in the subject by at least 1% (e.g., at least 5%, 10%, 15%, 20%, 25%, 30%, 35%, 40%, 45%, 50%, 55%, 60%, 65%, 70%, 75%, 80%, 85%, 90%, 95% or at least 98% or more; e.g., up to 99% or 100%) relative to the level of fat storage in the subject prior to the administering step. In some embodiments, the method reduces the blood cholesterol (e.g., LDL) level of the subject by at least 1% (e.g., at least 5%, 10%, 15%, 20%, 25%, 30%, 35%, 40%, 45%, 50%, 55%, or 60%; e.g., up to 70%, 80%, or 90%) relative to the blood cholesterol (e.g., LDL) level of the subject prior to the administering step. In some embodiments, the method reduces the hemoglobin A1c level of the subject by at least 1% (e.g., at least 5%, 10%, 15%, 20%, 25%, 30%, 35%, 40%, 45%, 50%, 55%, or 60%; e.g., up to 70%, 80%, or 90%) relative to the hemoglobin A1c level of the subject prior to the administering step. In some embodiments, the method reduces the blood triglycerides level of the subject by at least 1% (e.g., at least 5%, 10%, 15%, 20%, 25%, 30%, 35%, 40%, 45%, 50%, 55%, or 60%; e.g., up to 70%, 80%, or 90%) relative to the blood triglycerides level of the subject prior to the administering step.

In some embodiments, the method increases the insulin level in the subject by at least 1% (e.g., at least 5%, 10%, 15%, 20%, 25%, 30%, 35%, 40%, 45%, 50%, 55%, 60%, 65%, 70%, 75%, 80%, 85%, 90%, 95% or at least 98% or more; e.g., up to 99% or 100%) relative to the insulin level in the subject prior to the administering step. In some embodiments, the method increases the GLP-1 level in the subject by at least 1% (e.g., at least 5%, 10%, 15%, 20%, 25%, 30%, 35%, 40%, 45%, 50%, 55%, 60%, 65%, 70%, 75%, 80%, 85%, 90%, 95% or at least 98% or more; e.g., up to 99% or 100%) relative to the GLP-1 level in the subject prior to the administering step. In some embodiments, the method increases the PYY level in the subject by at least 1% (e.g., at least 5%, 10%, 15%, 20%, 25%, 30%, 35%, 40%, 45%, 50%, 55%, 60%, 65%, 70%, 75%, 80%, 85%, 90%, 95% or at least 98% or more; e.g., up to 99% or 100%) relative to the PYY level in the subject prior to the administering step. In some embodiments, the method increases the glucose tolerance in the subject by at least 1% (e.g., at least 5%, 10%, 15%, 20%, 25%, 30%, 35%, 40%, 45%, 50%, 55%, 60%, 65%, 70%, 75%, 80%, 85%, 90%, 95% or at least 98% or more; e.g., up to 99% or 100%) relative to the glucose tolerance in the subject prior to the administering step. In some embodiments, the method reduces the fasting blood sugar levels of the subject by at least 1% (e.g., at least 5%, 10%, 15%, 20%, 25%, 30%, 35%, or 40%; e.g., up to 50%) relative to the fasting blood sugar levels of the subject prior to the administering step. In some embodiments, the method reduces an elevated fasting blood sugar level in a subject to a normal fasting blood sugar level.

The markers described herein may be measured using methods known in the art. For example, glucose tolerance may be assessed using an oral glucose tolerance test (OGTT) described at MedlinePlus (medlineplus.gov). In this test, a subject drinks a liquid containing a predetermined amount of glucose (typically, 75 g of glucose), and blood glucose level is then measured at 15 minutes, 30 minutes, 60 minutes, 90 minutes, 120 minutes, 150 minutes, and 180 minutes after the glucose dosing. Insulin sensitivity can be measuring using an insulin clamp, for example, as described in Farrnnini and Mari, J. Hypertens., 16:895-906, 1998. Lipogenesis may be measured using a hepatic de novo lipogenesis test, for example, as described in Rabol et al., Proc. Nat. Acad. Sci., 108:13705-13709, 2011. This test assesses the incorporation of deuterium into plasma very-low-density lipoprotein triglyceride (VLDL) during administration of deuterium-labeled water.

Acylated active agents disclosed herein may be used in a method of treating a nonalcoholic fatty liver disease (e.g., nonalcoholic steatohepatitis (NASH) with or without fibrosis, liver steatosis, NASH with advanced fibrosis) in a subject in need thereof. Additionally or alternatively, acylated active agents disclosed herein may be used in a method of modulating a nonalcoholic fatty liver disease (e.g., nonalcoholic steatohepatitis) marker in a subject in need thereof.

Typically, the methods of treating NAFLD, e.g., NASH. or of modulating a NAFLD marker, e.g., NASH marker, include administration of acylated active agent disclosed herein to a subject in need thereof (e.g., a subject diagnosed with, or suffering from, NAFLD, e.g., NASH). In some embodiments, the components of the acylated active agent (e.g., a short chain fatty acid acyl (e.g., acetyl) in combination with a hydroxybenzoic acid, e.g., gentisic acid) may act synergistically to treat NAFLD (e.g., NASH) in a subject in need thereof. In certain embodiments, the components of the acylated active agent (e.g., a short chain fatty acid acyl (e.g., acetyl) in combination with a hydroxybenzoic acid, e.g., gentisic acid) may act synergistically to modulate a NAFLD marker in a subject in need thereof.

In some embodiments, the method reduces the level of alanine transaminase in the blood of the subject by at least 1% (e.g., at least 5%, 10%, 15%, 20%, 25%, 30%, 35%, 40%, 45%, 50%, 55%, 60%, 65%, 70%, 75%, 80%, 85%, 90%, 95% or at least 98% or more; e.g., up to 99% or 100%) relative to the level of alanine transaminase in the blood of the subject prior to the administering step. Certain methods disclosed herein may reduce the level of alanine transaminase in the blood of the subject to that which is considered normal for the subject (e.g., a human); a normal level of alanine transaminase in human blood is typically 7-56 units/L. In certain embodiments, the method reduces the level of aspartate transaminase in the blood of the subject by at least 1% (e.g., at least 5%, 10%, 15%, 20%, 25%, 30%, 35%, 40%, 45%, 50%, 55%, 60%, 65%, 70%, 75%, 80%, 85%, 90%, 95% or at least 98% or more; e.g., up to 99% or 100%) relative to the level of aspartate transaminase in the blood of the subject prior to the administering step. Certain methods disclosed herein may reduce the level of aspartate transaminase in the blood of the subject to that which is considered normal for the subject (e.g., a human); a normal level of aspartate transaminase in human blood is typically 10-40 units/L. In particular embodiments, the method reduces the liver weight of the subject by at least 1% relative to the liver weight of the subject prior to the administering step.

### Pharmaceutical and Nutraceutical Compositions

The active agents disclosed herein (e.g., an acylated hydroxybenzoic acid, or a pharmaceutically acceptable salt thereof, or an ester thereof) may be formulated into pharmaceutical or nutraceutical compositions for administration to human subjects in a biologically compatible form suitable for administration in vivo. Pharmaceutical and nutraceutical compositions typically include an active agent as described herein and a physiologically acceptable excipient (e.g., a pharmaceutically acceptable excipient). A nutraceutical composition may be, e.g., a dietary supplement or a food product, including an active agent disclosed herein (e.g., an acylated hydroxybenzoic acid, such as compound 3, a pharmaceutically acceptable salt thereof or an ester thereof).

The active agents described herein can also be used in the form of the free acid/base, in the form of salts, zwitterions, or as solvates. All forms are within the scope of the invention. The active agents, salts, zwitterions, solvates, or pharmaceutical or nutraceutical compositions thereof, may be administered to a subject in a variety of forms depending on the selected route of administration, as will be understood by those skilled in the art. The active agents described herein may be administered, for example, by oral, parenteral, buccal, sublingual, nasal, rectal, patch, pump, or transdermal administration, and the pharmaceutical or nutraceutical compositions formulated accordingly. Parenteral administration includes intravenous, intraperitoneal, subcutaneous, intramuscular, transepithelial, nasal, intrapulmonary, intrathecal, rectal, and topical modes of administration. Parenteral administration may be by continuous infusion over a selected period of time.

For human use, an active agent disclosed herein can be administered alone or in admixture with a pharmaceutical or nutraceutical carrier selected regarding the intended route of administration and standard pharmaceutical practice. Pharmaceutical and nutraceutical compositions for use in accordance with the present invention thus can be formulated in a conventional manner using one or more physiologically acceptable carriers comprising excipients and auxiliaries that facilitate processing of active agents disclosed herein into preparations which can be used pharmaceutically.

This disclosure also includes pharmaceutical and nutraceutical compositions which can contain one or more physiologically acceptable carriers. In making the pharmaceutical or nutraceutical compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semisolid, or liquid material (e.g., normal saline), which acts as a vehicle, carrier or medium for the active ingredient. Thus, the compositions can be in the form of tablets, powders, lozenges, sachets, cachets, elixirs, suspensions, emulsions, solutions, syrups, and soft and hard gelatin capsules. As is known in the art, the type of diluent can vary depending upon the intended route of administration. The resulting compositions can include additional agents, e.g., preservatives. Nutraceutical compositions may be administered internally (e.g., orally). A nutraceutical composition may be a nutraceutical oral formulation (e.g., a tablet, powder, lozenge, sachet, cachet, elixir, suspension, emulsion, solution, syrup, or soft or hard gelatin capsule), food additive (e.g., a food additive as defined in 21 C.F.R. § 170.3), food product (e.g., food for special dietary use as defined in 21 C.F.R. § 105.3), or dietary supplement [e.g., where the active agent is a dietary ingredient, e.g., as defined in 21 U.S.C. § 321(ff)]. Active agents can be used in nutraceutical applications and as food additive or food products. Non-limiting examples of compositions including an active agent of the invention are a bar, drink, shake, powder, additive, gel, or chew.

The excipient or carrier is selected on the basis of the mode and route of administration. Suitable pharmaceutical carriers, as well as pharmaceutical necessities for use in pharmaceutical formulations, are described in Remington: The Science and Practice of Pharmacy, 21^{st} Ed., Gennaro, Ed., Lippencott Williams & Wilkins (2005), a well-known reference text in this field, and in the USP/NF (United States Pharmacopeia and the National Formulary). Examples of suitable excipients are lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents, e.g., talc, magnesium stearate, and mineral oil; wetting agents; emulsifying and suspending agents; preserving agents, e.g., methyl- and propylhydroxy-benzoates; sweetening agents; and flavoring agents. Other exemplary excipients are described in Handbook of Pharmaceutical Excipients, 6^{th} Edition, Rowe et al., Eds., Pharmaceutical Press (2009).

These pharmaceutical and nutraceutical compositions can be manufactured in a conventional manner, e.g., by conventional mixing, dissolving, granulating, dragee-making, levigating, emulsifying, encapsulating, entrapping, or lyophilizing processes. Methods well known in the art for making formulations are found, for example, in Remington: The Science and Practice of Pharmacy, 21^{st} Ed., Gennaro, Ed., Lippencott Williams & Wilkins (2005), and Encyclopedia of Pharmaceutical Technology, eds. J. Swarbrick and J. C. Boylan, 1988-1999, Marcel Dekker, New York. Proper formulation is dependent upon the route of administration chosen. The formulation and preparation of such compositions is well-known to those skilled in the art of pharmaceutical and nutraceutical formulation. In preparing a formulation, the active agents can be milled to provide the appropriate particle size prior to combining with the other ingredients. If the active agent is substantially insoluble, it can be milled to a particle size of less than 200 mesh. If the active agent is substantially water soluble, the particle size can be adjusted by milling to provide a substantially uniform distribution in the formulation, e.g., about 40 mesh.

### Dosages

The dosage of the active agent used in the methods described herein, or pharmaceutically acceptable salts or prodrugs thereof, or pharmaceutical or nutraceutical compositions thereof, can vary depending on many factors, e.g., the pharmacodynamic properties of the active agent; the mode of administration; the age, health, and weight of the recipient; the nature and extent of the symptoms; the frequency of the treatment, and the type of concurrent treatment, if any; and the clearance rate of the active agent in the subject to be treated. One of skill in the art can determine the appropriate dosage based on the above factors. The active agents used in the methods described herein may be administered initially in a suitable dosage that may be adjusted as required, depending on the clinical response. In general, a suitable daily dose of an active agent disclosed herein will be that amount of the active agent that is the lowest dose effective to produce a therapeutic effect. Such an effective dose will generally depend upon the factors described above.

An active agent disclosed herein may be administered to the subject in a single dose or in multiple doses. When multiple doses are administered, the doses may be separated from one another by, for example, 1-24 hours, 1-7 days, or 1-4 weeks. The active agent may be administered according to a schedule, or the active agent may be administered without a predetermined schedule. It is to be understood that, for any particular subject, specific dosage regimes should be adjusted over time according to the individual need and the professional judgment of the person administering or supervising the administration of the compositions.

The active agents may be provided in a unit dosage form. In some embodiments, the unit dosage form may be an oral unit dosage form (e.g., a tablet, capsule, suspension, liquid solution, powder, crystals, lozenge, sachet, cachet, elixir, syrup, and the like) or a food product serving (e.g., the active agents may be included as food additives or dietary ingredients). In certain embodiments, the unit dosage form is designed for administration of an acylated active agent disclosed herein, where the total amount of an administered acylated active agent(s) is from 0.1 g to 10 g (e.g., 0.5 g to 9 g, 0.5 g to 8 g, 0.5 g to 7 g, 0.5 g to 6 g, 0.5 g to 5 g, 0.5 g to 1 g, 0.5 g to 1.5 g, 0.5 g to 2 g, 0.5 g to 2.5 g, 1 g to 1.5 g, 1 g to 2 g, 1 g to 2.5 g, 1.5 g to 2 g, 1.5 g to 2.5 g, or 2 g to 2.5 g). In other embodiments, the acylated active agent is consumed at a rate of 0.1 g to 10 g per day (e.g., 0.5 g to 9 g, 0.5 g to 8 g, 0.5 g to 7 g, 0.5 g to 6 g, 0.5 g to 5 g, 0.5 g to 1 g per day, 0.5 g to 1.5 g per day, 0.5 g to 2 g per day, 0.5 g to 2.5 g per day, 1 g to 1.5 g per day, 1 g to 2 g per day, 1 g to 2.5 g per day, 1.5 g to 2 g per day, 1.5 g to 2.5 g per day, or 2 g to 2.5 g per day) or more. The attending physician ultimately will decide the appropriate amount and dosage regimen, an effective amount of the active agent disclosed herein may be, for example, a total daily dosage of, e.g., between 0.5 g and 10 g (e.g., 0.5 to 5 g) of any of the acylated active agent described herein. Alternatively, the dosage amount can be calculated using the body weight of the subject. Preferably, when daily dosages exceed 5 g/day, the dosage of the active agents may be divided across two or three daily administration events.

In the methods of the invention, the time period during which multiple doses of an active agent disclosed herein are administered to a subject can vary. For example, in some embodiments doses of the active agents are administered to a subject over a time period that is 1-7 days; 1-12 weeks; or 1-3 months. In other embodiments, the active agents are administered to the subject over a time period that is, for example, 4-11 months or 1-30 years. In yet other embodiments, the active agents disclosed herein are administered to a subject at the onset of symptoms. In any of these embodiments, the amount of the active agent that is administered may vary during the time period of administration. When an active agent is administered daily, administration may occur, for example, 1, 2, 3, or 4 times per day.

### Formulations

An active agent described herein may be administered to a subject with a pharmaceutically acceptable diluent, carrier, or excipient, in unit dosage form. Administration may begin before the subject is symptomatic.

Exemplary routes of administration of the active agents disclosed herein or pharmaceutical or nutraceutical compositions thereof, used in the present invention include oral, sublingual, buccal, transdermal, intradermal, intramuscular, parenteral, intravenous, intra-arterial, intracranial, subcutaneous, intraorbital, intraventricular, intraspinal, intraperitoneal, intranasal, inhalation, and topical administration. The active agents desirably are administered with a physiologically acceptable carrier (e.g., a pharmaceutically acceptable carrier). Pharmaceutical formulations of the active agents described herein formulated for treatment of the disorders described herein are also part of the present invention. In some preferred embodiments, the active agents disclosed herein are administered to a subject orally.

### Formulations for Oral Administration

The pharmaceutical and nutraceutical compositions contemplated by the invention include those formulated for oral administration (“oral unit dosage forms”). Oral unit dosage forms can be, for example, in the form of tablets, capsules, a liquid solution or suspension, a powder, or liquid or solid crystals, which contain the active ingredient(s) in a mixture with physiologically acceptable excipients (e.g., pharmaceutically acceptable excipients). These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other physiologically acceptable excipients (e.g., pharmaceutically acceptable excipients) can be colorants, flavoring agents, plasticizers, humectants, buffering agents, and the like.

Formulations for oral administration may also be presented as chewable tablets, as hard gelatin capsules where the active ingredient is mixed with an inert solid diluent (e.g., potato starch, lactose, microcrystalline cellulose, calcium carbonate, calcium phosphate or kaolin), or as soft gelatin capsules where the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Controlled release compositions for oral use may be constructed to release the active drug by controlling the dissolution and/or the diffusion of the active drug substance. Any of a number of strategies can be pursued in order to obtain controlled release and the targeted plasma concentration versus time profile. In one example, controlled release is obtained by appropriate selection of various formulation parameters and ingredients, including, e.g., various types of controlled release compositions and coatings. Examples include single or multiple unit tablet or capsule compositions, oil solutions, suspensions, emulsions, microcapsules, microspheres, nanoparticles, patches, and liposomes. In certain embodiments, compositions include biodegradable, pH, and/or temperature-sensitive polymer coatings.

Dissolution- or diffusion-controlled release can be achieved by appropriate coating of a tablet, capsule, pellet, or granulate formulation of active agents, or by incorporating the active agent into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

The liquid forms in which the active agents and compositions of the present invention can be incorporated for administration orally include aqueous solutions, suitably flavored syrups, aqueous or oil suspensions, and flavored emulsions with edible oils, e.g., cottonseed oil, sesame oil, coconut oil, or peanut oil, as well as elixirs and similar pharmaceutical and nutraceutical vehicles.

### Formulations for Buccal Administration

Dosages for buccal or sublingual administration typically are 0.1 to 500 mg per single dose as required. In practice, the physician determines the actual dosing regimen which is most suitable for an individual subject, and the dosage varies with the age, weight, and response of the particular subject. The above dosages are exemplary of the average case, but individual instances exist where higher or lower dosages are merited, and such are within the scope of this invention.

For buccal administration, the compositions may take the form of tablets, lozenges, etc. formulated in a conventional manner. Liquid drug formulations suitable for use with nebulizers and liquid spray devices and electrohydrodynamic (EHD) aerosol devices will typically include a active agent disclosed herein with a pharmaceutically acceptable carrier. Preferably, the pharmaceutically acceptable carrier is a liquid, e.g., alcohol, water, polyethylene glycol, or a perfluorocarbon. Optionally, another material may be added to alter the aerosol properties of the solution or suspension of active agents disclosed herein. Desirably, this material is liquid, e.g., an alcohol, glycol, polyglycol, or a fatty acid. Other methods of formulating liquid drug solutions or suspension suitable for use in aerosol devices are known to those of skill in the art (see, e.g., U.S. Pat. Nos. 5,112,598 and 5,556,611, each of which is herein incorporated by reference).

### Formulations for Nasal or Inhalation Administration

The active agents may also be formulated for nasal administration. Compositions for nasal administration also may conveniently be formulated as aerosols, drops, gels, and powders. The formulations may be provided in a single or multidose form. In the case of a dropper or pipette, dosing may be achieved by the subject administering an appropriate, predetermined volume of the solution or suspension. In the case of a spray, this may be achieved, for example, by means of a metering atomizing spray pump.

The active agents may further be formulated for aerosol administration, particularly to the respiratory tract by inhalation and including intranasal administration. The active agents for nasal or inhalation administration will generally have a small particle size for example on the order of five (5) microns or less. Such a particle size may be obtained by means known in the art, for example by micronization. The active ingredient is provided in a pressurized pack with a suitable propellant, e.g., a chlorofluorocarbon (CFC), for example, dichlorodifluoromethane, trichlorofluoromethane, or dichlorotetrafluoroethane, or carbon dioxide, or other suitable gas. The aerosol may conveniently also contain a surfactant, e.g., lecithin. The dose of drug may be controlled by a metered valve. Alternatively, the active ingredients may be provided in a form of a dry powder, e.g., a powder mix of the active agent in a suitable powder base, e.g., lactose, starch, and starch derivatives, e.g., hydroxypropylmethyl cellulose, and polyvinylpyrrolidine (PVP). The powder carrier will form a gel in the nasal cavity. The powder composition may be presented in unit dose form for example in capsules or cartridges of e.g., gelatin or blister packs from which the powder may be administered by means of an inhaler.

Aerosol formulations typically include a solution or fine suspension of the active substance in a physiologically acceptable aqueous or non-aqueous solvent and are usually presented in single or multidose quantities in sterile form in a sealed container, which can take the form of a cartridge or refill for use with an atomizing device. Alternatively, the sealed container may be a unitary dispensing device, e.g., a single dose nasal inhaler or an aerosol dispenser fitted with a metering valve which is intended for disposal after use. Where the unit dosage form comprises an aerosol dispenser, it will contain a propellant, which can be a compressed gas, e.g., compressed air or an organic propellant, e.g., fluorochlorohydrocarbon. The aerosol unit dosage forms can also take the form of a pump-atomizer.

### Formulations for Parenteral Administration

The active agents described herein for use in the methods of the invention can be administered in a pharmaceutically acceptable parenteral (e.g., intravenous or intramuscular) formulation as described herein. The pharmaceutical formulation may also be administered parenterally (intravenous, intramuscular, subcutaneous or the like) in unit dosage forms or formulations containing conventional, non-toxic pharmaceutically acceptable carriers and adjuvants. In particular, formulations suitable for parenteral administration include aqueous and non-aqueous sterile injection solutions which may contain anti-oxidants, buffers, bacteriostats, and solutes which render the formulation isotonic with the blood of the intended recipient; and aqueous and non-aqueous sterile suspensions which may include suspending agents and thickening agents. For example, to prepare such a composition, the active agents disclosed herein may be dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution and isotonic sodium chloride solution. The aqueous formulation may also contain one or more preservatives, for example, methyl, ethyl or n-propyl p-hydroxybenzoate. Additional information regarding parenteral formulations can be found, for example, in the United States Pharmacopeia-National Formulary (USP-NF), herein incorporated by reference.

The parenteral formulation can be any of the five general types of preparations identified by the USP-NF as suitable for parenteral administration:
(1) “Drug Injection:” a liquid preparation that is a drug substance (e.g., an active agent disclosed herein or a solution thereof);
(2) “Drug for Injection:” the drug substance (e.g., an active agent disclosed herein) as a dry solid that will be combined with the appropriate sterile vehicle for parenteral administration as a drug injection;
(3) “Drug Injectable Emulsion:” a liquid preparation of the drug substance (e.g., an active agent disclosed herein) that is dissolved or dispersed in a suitable emulsion medium;
(4) “Drug Injectable Suspension:” a liquid preparation of the drug substance (e.g., an active agent disclosed herein) suspended in a suitable liquid medium; and
(5) “Drug for Injectable Suspension:” the drug substance (e.g., an active agent disclosed herein) as a dry solid that will be combined with the appropriate sterile vehicle for parenteral administration as a drug injectable suspension.

Exemplary formulations for parenteral administration include solutions of the active agents prepared in water suitably mixed with a surfactant, e.g., hydroxypropylcellulose. Dispersions can also be prepared in glycerol, liquid polyethylene glycols, DMSO and mixtures thereof with or without alcohol, and in oils. Under ordinary conditions of storage and use, these preparations may contain a preservative to prevent the growth of microorganisms. Conventional procedures and ingredients for the selection and preparation of suitable formulations are described, for example, in Remington: The Science and Practice of Pharmacy, 21^{st} Ed., Gennaro, Ed., Lippencott Williams & Wilkins (2005) and in The United States Pharmacopeia: The National Formulary (USP 36 NF31), published in 2013.

Formulations for parenteral administration may, for example, contain excipients, sterile water, or saline, polyalkylene glycols, e.g., polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the active agents or biologically active agents within active agents. Other potentially useful parenteral delivery systems for active agents include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

The parenteral formulation can be formulated for prompt release or for sustained/extended release of the active agent. Exemplary formulations for parenteral release of the active agent include: aqueous solutions, powders for reconstitution, cosolvent solutions, oil/water emulsions, suspensions, oil-based solutions, liposomes, microspheres, and polymeric gels.

### Preparation of Acylated Active Agents

Acylated active agents may be prepared using synthetic methods and reaction conditions known in the art. Optimum reaction conditions and reaction times may vary depending on the reactants used. Unless otherwise specified, solvents, temperatures, pressures, and other reaction conditions may be selected by one of ordinary skill in the art.

### Ester Preparation Strategy #1 (Acylation)

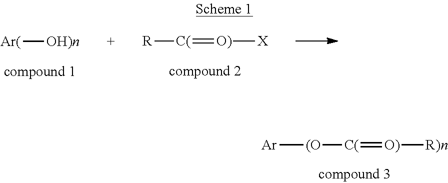

In Scheme 1, a phenolic compound, compound 1 where n represents an integer from 1 to 15, is treated with an acylating agent, compound 2, in an appropriate solvent, optionally in the presence of a catalyst. Suitable catalysts include pyridine, dimethylaminopyridine, trimethylamine and the like. The catalyst can be used in quantities ranging from 0.01 to 1.1 equivalents, relative to compound 2. Suitable solvents include methylene chloride, ethyl acetate, diethyl ether, tetrahydrofuran, 1,4-dioxane, 1,2-dimethoxyethane, toluene, combinations thereof and the like. Reaction temperatures range from −10° C. to the boiling point of the solvent used; reaction completion times range from 1 to 96 h. Suitable acylating agents include acyl chlorides, acyl fluorides, acyl bromides, carboxylic acid anhydrides whether symmetrical or not. A suitable acylating agent may also be generated in situ by prior reaction of a carboxylic acid with an activating reagent such as EDC or EEDQ or the like. The acylating agents can be used in quantities ranging from 0.5 to 15 equivalents relative to compound 1.

The product, compound 3, can be purified by methods known to those of skill in the art.

### Ester Preparation Strategy #2 (Acylation)

In some cases, the phenolic compound 1 may contain a functional group, Y, required to remain unreacted in the course of ester formation. In this case, it is appropriate to protect the functional group, Y, in the phenolic compound from acylation. This functional group may be an amino group or a hydroxyl group or other functionality with a labile hydrogen attached to a heteroatom. Such phenol esters can be prepared according to Scheme 2.

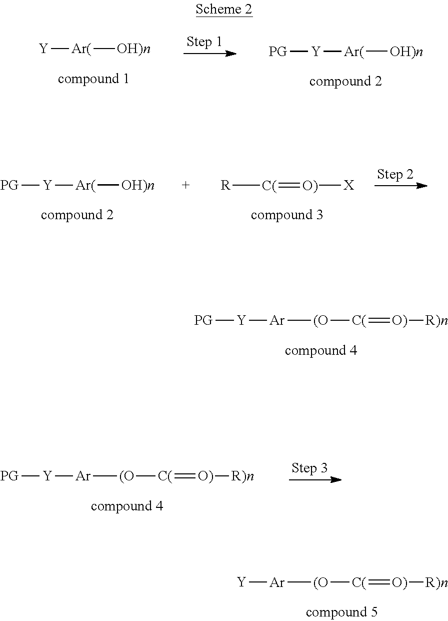

In Scheme 2 Step 1, compound 1, a phenolic compound containing a functional group Y with a labile hydrogen in need of protection, is treated with a protecting reagent such as BOC anhydride, benzyoxycarbonyl chloride, FMOC chloride, benzyl bromide and the like in an appropriate solvent, optionally in the presence of a catalyst to provide compound 2 scheme 2. Compound 2 can be purified by methods known to those of skill in the art.

In Scheme 2 Step 2, compound 2 is treated with an acylating agent, compound 3, in an appropriate solvent, optionally in the presence of a catalyst. Suitable catalysts include pyridine, dimethylaminopyridine, trimethylamine and the like. The catalyst can be used in quantities ranging from 0.01 to 1.1 equivalents, relative to compound 2. Suitable solvents include methylene chloride, ethyl acetate, diethyl ether, tetrahydrofuran, 1,4-dioxane, 1,2-dimethoxyethane, toluene, combinations thereof and the like. Reaction temperatures range from −10° C. to the boiling point of the solvent used; reaction completion times range from 1 to 96 h. Suitable acylating agents include acyl chlorides, acyl fluorides, acyl bromides, carboxylic acid anhydrides whether symmetrical or not. A suitable acylating agent may also be generated in situ by prior reaction of a carboxylic acid with an activating reagent such as EDC or EEDQ or the like. The acylating agents can be used in quantities ranging from 0.5 to 15 equivalents, relative to compound 3. Compound 4 can be purified by methods known to those of skill in the art.

In Scheme 2 Step 3, compound 4 is subjected to conditions that cleave the protecting group, PG.

In the case of a BOC protecting group, the protecting group of compound 4 is removed under acidic conditions to give compound 5 of the invention. Suitable acids include trifluoroacetic acid, hydrochloric acid, p-toluenesulfonic acid and the like.

In the case of an FMOC protecting group, the protecting group of compound 4 is removed under basic conditions to give compound 5 of the invention. Suitable bases include piperidine, triethylamine and the like. Suitable solvents include DMF, NMP dichloromethane and the like. The FMOC group is also removed under non-basic conditions such as by treatment with tetrabutylammonium fluoride trihydrate in a suitable solvent such as DMF. The FMOC group is also removed by catalytic hydrogenation. Suitable catalysts for hydrogenation include 10% palladium-on-charcoal and palladium (II) acetate and the like. Suitable solvents for hydrogenation include DMF, ethanol, and the like

In the case of a benzyloxycarbonyl or benzyl protecting group the protecting group of compound 4 is removed by hydrogenation to give compound 5. Suitable catalysts for hydrogenation include 10% Palladium-on-charcoal and Palladium acetate and the like. Suitable solvents for hydrogenation include DMF, ethanol, methanol, ethyl acetate, and the like. The product, compound 5, can be purified by methods known to those of skill in the art.

### Ester Preparation Strategy #3 (Acylation)

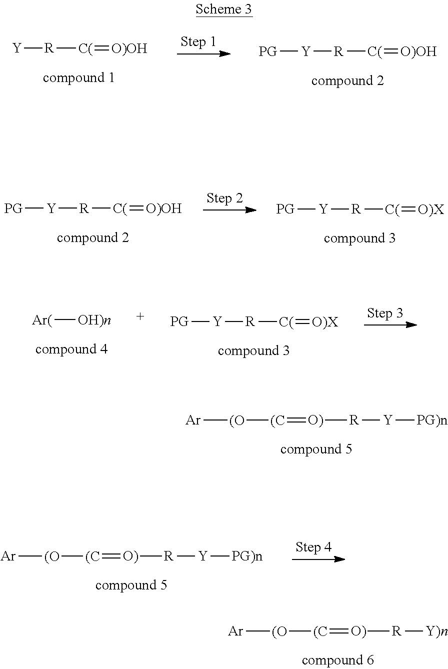

In Scheme 3 Step 1, compound 1, an acyl compound containing a functional group Y with a labile hydrogen in need on protection, is treated with a protecting reagent such as BOC anhydride, benzyoxycarbonyl chloride, FMOC chloride, benzyl bromide and the like in an appropriate solvent, optionally in the presence of a catalyst to provide compound 2 scheme 3. Compound 2 can be purified by methods known to those of skill in the art.

In Scheme 3 Step 2, compound 2 is treated with an activating reagent such as thionyl chloride, phosphorus oxychloride, EDC or EEDQ or the like to generate the activated acyl compound 3.

In Scheme 3 Step 3, the phenol compound 4 is treated with the activated acyl compound 3, in an appropriate solvent, optionally in the presence of a catalyst. Suitable catalysts include pyridine, dimethylaminopyridine, trimethylamine and the like to generate compound 5. The catalyst can be used in quantities ranging from 0.01 to 1.1 equivalents, relative to compound 3. Suitable solvents include methylene chloride, ethyl acetate, diethyl ether, tetrahydrofuran, 1,4-dioxane, 1,2-dimethoxyethane, toluene, combinations thereof and the like. Reaction temperatures range from −10° C. to the boiling point of the solvent used; reaction completion times range from 1 to 96 h. The activated acyl compound 3 can be used in quantities ranging from 0.5 to 15 equivalents relative to compound 4.

In Scheme 3 Step 4, compound 5 is subjected to conditions designed to cleave the protecting group, PG, illustrated in Scheme 2 above. The product, compound 6, can be purified by methods known to those of skill in the art.

### Ester preparation strategy #4 (Nucleophilic Alkylation)

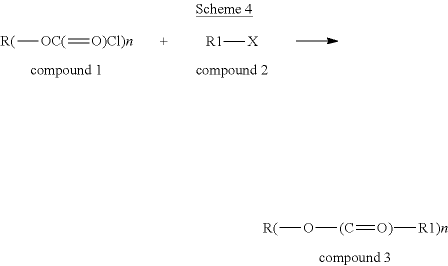

In Scheme 4 Step 1, a chloroformate compound, compound 1, where R represents an aromatic moiety or a non-aromatic cyclic or acyclic moiety, is treated, in an appropriate solvent, with an organometallic compound, compound 2 where R1 represents an alkyl group optionally substituted with one or more protected hydroxyl groups and X represents a metal such as Cu, Zn, Mg which is optionally coordinated by one or more counterions, such as chloride. Suitable solvents include methylene chloride, THF, acetonitrile, toluene, diethyl ether, combinations thereof, and the like. Reaction temperatures range from −10° C. to the boiling point of the solvent used; reaction completion times range from 1 to 96 h. The product, compound 3, can be purified by methods known to those of skill in the art.

Compound 1 can be prepared from the corresponding alcohol or polyol compounds by standard methods familiar to one skilled in the art.

Where compound 2 is optionally substituted by one or more protected alcohol groups deprotection is accomplished by the methods illustrated in Scheme 2 above.

Further modification of the initial product by methods known in the art and illustrated in the examples below, may be used to prepare additional compounds of this invention.

### Ester Preparation Strategy #5 (Acylation)

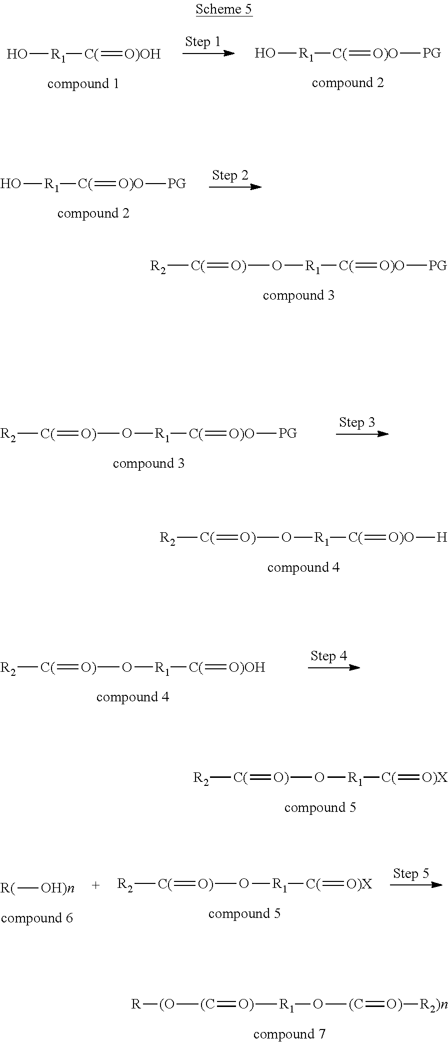

In Scheme 5 Step 1, compound 1, an acyl compound containing a hydroxyl group to be acylated, is treated with a protecting reagent such as benzyl bromide and the like in an appropriate solvent, optionally in the presence of a catalyst to provide compound 2 scheme 5. Compound 2 can be purified by methods known to those of skill in the art.

In scheme 5 Step 2, compound 2 is treated with an acylating agent, in an appropriate solvent, optionally in the presence of a catalyst. Suitable catalysts include pyridine, dimethylaminopyridine, trimethylamine and the like. The catalyst can be used in quantities ranging from 0.01 to 1.1 equivalents, relative to compound 2. Suitable solvents include methylene chloride, ethyl acetate, diethyl ether, tetrahydrofuran, 1,4-dioxane, 1,2-dimethoxyethane, toluene, combinations thereof and the like. Reaction temperatures range from −10° C. to the boiling point of the solvent used; reaction completion times range from 1 to 96 h. Suitable acylating agents include acyl chlorides, acyl fluorides, acyl bromides, carboxylic acid anhydrides whether symmetrical or not. A suitable acylating agent may also be generated in situ by a reaction of a carboxylic acid with an activating reagent such as EDC or EEDQ or the like. The acylating agents can be used in quantities ranging from 0.5 to 15 equivalents relative to compound 1.

In Scheme 5 Step 3, compound 3 is subjected to conditions that cleave the protecting group, PG. In the case of a benzyl protecting group, the protecting group of compound 3 is removed by hydrogenation to give compound 4. Suitable catalysts for hydrogenation include 10% palladium-on-charcoal and palladium acetate and the like. Suitable solvents for hydrogenation include, DMF, ethanol, methanol, ethyl acetate and the like. The product, compound 4, can be purified by methods known to those of skill in the art.

In Scheme 5 Step 4, compound 4 is treated with an activating reagent such as thionyl chloride, phosphorus oxychloride, EDC or EEDQ or the like to generate the activated acyl compound 5.

In Scheme 5 Step 5, the poly-hydroxyl compound, compound 6, where R represents an aromatic or an aliphatic cyclic or acyclic core, is treated with the activated acyl compound 5, in an appropriate solvent, optionally in the presence of a catalyst. Suitable catalysts include pyridine, dimethylaminopyridine, trimethylamine and the like to generate compound 5. The catalyst can be used in quantities ranging from 0.01 to 1.1 equivalents, relative to compound 3. Suitable solvents include methylene chloride, ethyl acetate, diethyl ether, tetrahydrofuran, 1,4-dioxane, 1,2-dimethoxyethane, toluene, combinations thereof and the like. Reaction temperatures range from -10° C. to the boiling point of the solvent used; reaction completion times range from 1 to 96 h. The activated acyl compound 5 can be used in quantities ranging from 0.5 to 15 equivalents relative to compound 6.

The product, compound 7, can be purified by methods known in the art.

The following examples are meant to illustrate the invention. They are not meant to limit the invention in any way.

### EXAMPLES

### Example 1. Preparation of Exemplary Acylated Active Agents

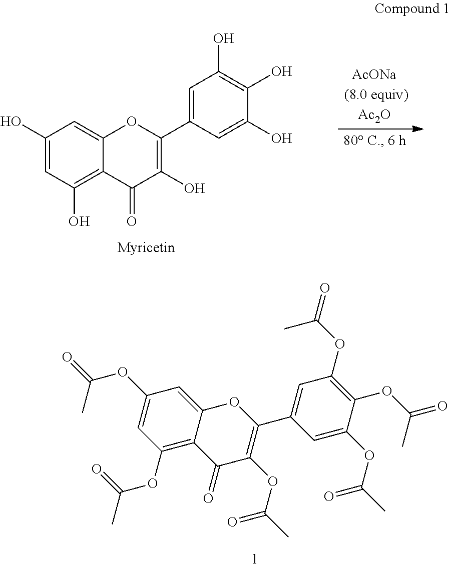 [SMILES: O=c1c(O)c(-c2cc(O)c(O)c(O)c2)oc2cc(O)cc(O)c12 ; CC(=O)Oc1cc(OC(C)=O)c2c(=O)c(OC(C)=O)c(-c3cc(OC(C)=O)c(OC(C)=O)c(OC(C)=O)c3)oc2c1]

Six reactions were carried out in parallel. To a solution of myricetin (330 g, 1.04 mol, 1.0 eq) in Ac₂O (2 L) was added AcONa (681 g, 8.30 mol, 8.0 equiv.). The suspension was stirred at 80° C. for 6 h. TLC (petroleum ether/ethyl acetate=2/1, R_{f}=0.7) showed the reaction was completed. The six reactions were combined for the work up. The reaction solution was poured into ice-water (30 L) and stirred for 2 h to give a precipitate, which was collected by filtration. The crude product was triturated with ethyl acetate (10 L) at 25° C. for 1 h. The suspension was filtered, and the filter cake was dried under reduced pressure to give compound 1 (2.0 kg, 56.4% yield) as a white solid. ¹H NMR: (400 MHz, CDCl₃) δ 7.62 (s, 2H), 7.34 (d, J=2.0 Hz, 1H), 6.88 (d, J=3.2 Hz, 1H), 2.44 (s, 3H), 2.37 (s, 3H), 2.35 (s, 3H), 2.34 (s, 3H), 2.33 (s, 6H) ppm.

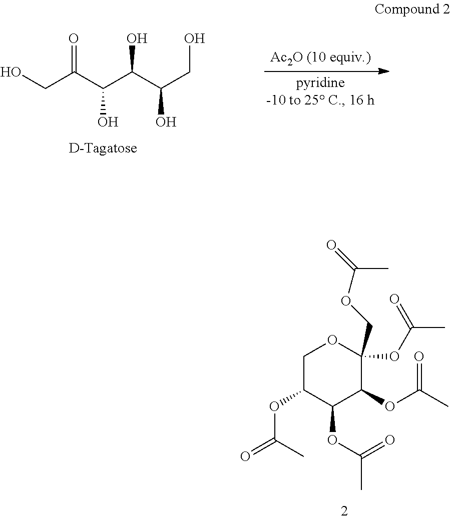 [SMILES: O=C(CO)[C@@H](O)[C@@H](O)[C@H](O)CO ; CC(=O)OC[C@]1(OC(C)=O)OC[C@@H](OC(C)=O)[C@H](OC(C)=O)[C@@H]1OC(C)=O]

To a solution of D-tagatose (200 g, 1.11 mol, 1.0 equiv.) in pyridine (1.6 L) was added AC₂O (1.13 kg, 11.1 mol, 1.04 L, 10 equiv.) dropwise at −10° C. under Nz. After addition, the suspension was stirred at 25° C. for 16 h. TLC (petroleum ether/ethyl acetat=2/1, R_{f}=0.45) showed the reaction was completed. The reaction solution was poured into ice-water (5.0 L), and then extracted with EtOAc (3.0 L, 2.0 L). The combined organic layers were washed with HCl (1.0 M, 1.0 L×2), brine (1.0 L), dried over NazSat, filtered, and concentrated under reduced pressure to give a residue. The residue was purified by column chromatography (SiO₂, petroleum ether/ethyl acetate=10/1 to 1/1) to give compound 2 (100 g, 256 mmol, 23.1% yield) as a white solid. ¹H NMR: (400 MHz, CDCl₃) δ 5.47 (d, J=3.6 Hz, 1H), 5.35 (dd, J=10.4, 3.2 Hz, 1H), 5.22-5.29 (m, 1H), 4.80 (d, J=12.0 Hz, 1H), 4.42 (d, J=12.0 Hz, 1H), 4.11 (dd, J=11.2, 6.0 Hz, 1H), 3.51 (t, J=10.8 Hz, 1H), 2.17 (s, 3H), 2.14 (s, 3H), 2.06 (s, 3H), 2.03 (s, 3H), 2.01 (s, 3H) ppm.

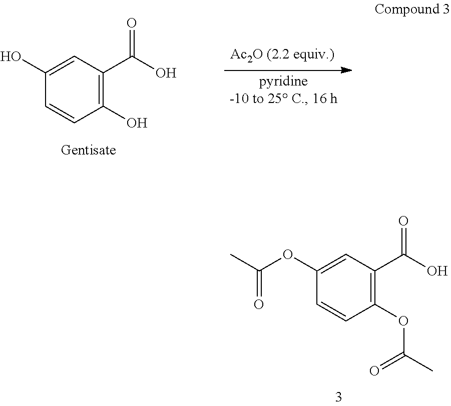 [SMILES: O=C(O)c1cc(O)ccc1O ; CC(=O)Oc1ccc(OC(C)=O)c(C(=O)O)c1]

Five reactions were carried out in parallel. To a suspension of gentisate (500 g, 3.24 mol, 1.0 equiv.) in pyridine (3.0 L) was added Ac₂O (729 g, 7.14 mol, 668 mL, 2.2 equiv.) dropwise at −10° C. under Nz. The suspension was stirred at 25° C. for 16 h. TLC (petroleum ether/ethyl acetate=3/1, R_{f}=0.5) showed the reaction was completed. The five reactions were combined for work up. The reaction solution was poured into ice-water mixture (7.0 L), and then extracted with EtOAc (3.0 L, 2.0 L). The combined organic layers were washed with 1 M HCl (2.0 L) twice. Then, the organic phase was washed with brine (2.0 L), dried over NazSat, filtered and concentrated under reduced pressure to give a residue. The residue was triturated with MTBE (3 v) at 25° C. for 2 h. The suspension was filtered, and the filter cake was collected and dried under reduced pressure to give compound 3 (2.0 kg, 8.40 mol, 51.8% yield) as a white solid. ¹H NMR: (400 MHz, DMSO-d6) δ 13.9 (s, 1H), 7.67 (d, J=2.8 Hz, 1H), 7.41 (dd, J=8.4, 2.8 Hz, 1H), 7.25 (d, J=8.8 Hz, 1H), 2.28 (s, 3H), 2.25 (s, 3H) ppm.

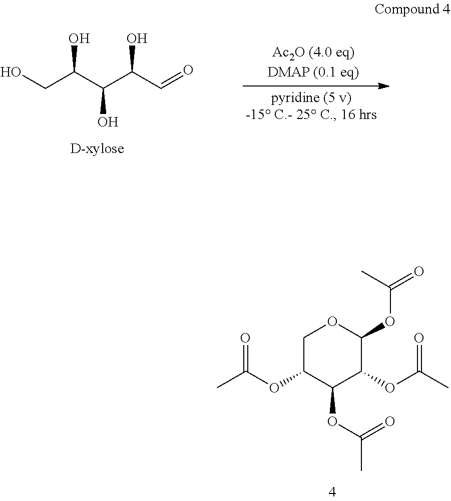 [SMILES: O=C[C@H](O)[C@@H](O)[C@H](O)CO ; CC(=O)O[C@@H]1OC[C@@H](OC(C)=O)[C@H](OC(C)=O)[C@H]1OC(C)=O]

To a solution of D-xylose (500 g, 3.33 mol, 1.0 eq) in AC₂O (4 L) was added AcONa (273 g, 3.33 mol, 1.0 eq). After addition, the resulting suspension was stirred at 80° C. for 6 hrs. TLC (petroleum ether/ethyl acetate=5/1, R_{f}=0.75) showed the reaction was complete. Four parallel reactions were combined for work up. The reaction solution was poured into ice-water (30 L) and stirred for 2 h. Ample solids precipitated and were collected by filtration. The residue was triturated with H₂O (10.0 L) at 25° C. for 3 hrs. The suspension was filtered, and the filter cake was collected and dried under reduced pressure to give compound 4 (2.0 kg, 47.2% yield) as a white solid. ¹H NMR: (400 MHz, CDCl₃) δ 5.71 (d, J=7.2 Hz, 1H), 5.20 (t, J=8.0 Hz, 1H), 4.97-5.05 (m, 2H), 4.14 (dd, J=12.0, 4.8 Hz, 1H), 3.53 (q, J=12.0 Hz, 1H), 2.11(s, 3H), 2.06 (s, 3H), 2.05 (s, 6H) ppm.

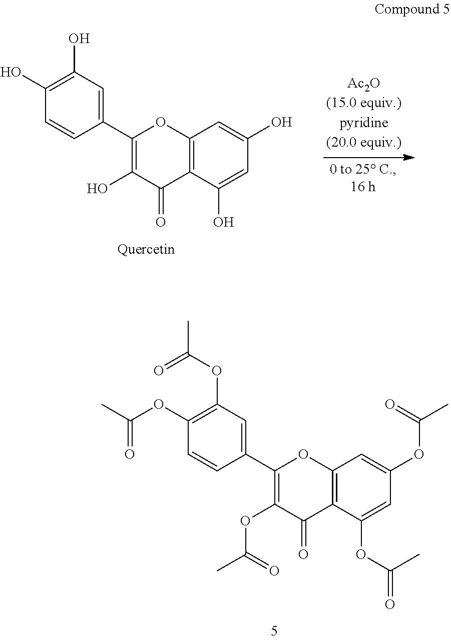 [SMILES: O=c1c(O)c(-c2ccc(O)c(O)c2)oc2cc(O)cc(O)c12 ; CC(=O)Oc1cc(OC(C)=O)c2c(=O)c(OC(C)=O)c(-c3ccc(OC(C)=O)c(OC(C)=O)c3)oc2c1]

Eight reactions were carried out in parallel. To a solution of quercetin (300 g, 992 mmol, 1.00 equiv.) in pyridine (1.60 L) was added dropwise acetyl acetate (1.52 kg, 14.9 mol, 1.39 L, 15.0 equiv.) at 0° C. After addition, the mixture was stirred at 25° C. for 16 h. TLC (dichloromethane/methanol=10/1, R_{f}=0.63) indicated complete consumption of quercetin. The eight reaction mixtures were combined, poured into ice-water (w/w=1/1, 24.0 L) and stirred for 1 h. The suspensions were filtered to give a yellow solid. The solid was dried under vacuum and was combined with another batch of compound 5 (300 g). The combined crude product was dissolved in MeCN (10.0 L) and heated to 65° C. EtOH (12.0 L) was added drop-wise at 65° C., and then the suspension was stirred at 65° C. for 1 h. White solid formed and was filtered. The filter cake was rinsed with EtOH (2.00 L), collected and dried under vacuum (40° C., −0.09 MPa) to give the compound 5 (2005 g, 3.91 mol, 39.3% total yield) as a white solid. ¹H NMR (400 MHz, CDCl₃): δ 7.68-7.73 (m, 2H), 7.33-7.36 (m, 2H), 6.87 (d, J=2.0 Hz, 1H), 2.43 (s, 3H), 2.33-2.34 (m, 12H) ppm.

### Example 2. Mouse Adipocyte Lipolysis Assay

Mouse 3T3-L1 cells were obtained from ATCC and cultured in Dulbecco's Modified Eagle's medium (DMEM) containing 10% newborn calf serum (NCS) and penicillin/streptomycin(P/S) at 37° C. in an incubator with 5% CO₂. Once the cells became confluent, they were seeded into a tissue culture treated 96 well plate. Then, differentiation was initiated by using DMEM containing 10% fetal bovine serum, P/S, IBMX, dexamethasone, and insulin. After 14 days of differentiation, cells were treated with compounds of interest. After 24 hours post-treatment, the cell viability was assessed using CellTiter-Glo Luminescent Cell Viability Assay from Promega, and lipolysis was determined using Lipolysis Assay Kit from ZenBio. No treatment had a significant effect on cell viability (>90% of DMSO control).

TABLE 1

Lipolysis

| | Free Fatty Acids | Glycerol |
|---|---|---|
| | % DMSO | % change |
| Acetic acid 1 mM | ++ | + |
| Acetic acid 3 mM | ++ | + |
| Butyric acid 3 mM | ++ | ++ |
| Propionic acid 3 mM | +++ | ++ |
| DMSO | = (100%) | = (100%) |

90% > ≤100%: =
70% > ≤90%: +
50 > ≤70%: ++
50%≤: +++

Table 1 lists the compounds that reduced the release of free fatty acids and glycerol (+, ++, or +++). The lipolytic rate of white adipose tissue is associated with metabolic dysfunction including insulin resistance and liver steatosis. Compounds that lower lipolysis of adipocytes may improve metabolic function including improving insulin sensitivity and reducing liver steatosis, thus improving outcomes in subjects with diabetes mellitus (e.g., prediabetes or type II diabetes), obesity, and hyperlipidemia.

### Example 3. Mouse Myocyte Lipolysis Assay

Cells were obtained from ATCC and cultured in Dulbecco's Modified Eagle's medium (DMEM) containing 20% fetal bovine serum and 1% penicillin/streptomycin at 37° C. in an incubator with 5% CO₂ Once the cells became confluent, they were seeded into a tissue culture treated 96 well plate. The next day, the medium containing DMEM with 2% equine serum was used to start differentiation. Once cells were fully differentiated, they were treated with compounds listed in Table 2.

TABLE 2

| | Free glycerol |
|---|---|
| Treatment | % DMSO |
| Acetic acid 1 mM | = |
| Acetic acid 3 mM | = |
| Butyric acid 3 mM | +++ |
| Propionic acid 3 mM | ++ |
| Gentisate 100 μM | +++ |
| DMSO | = (100.0%) |

90% > <100%: =
70% > <90%: +
50 > <70%: ++
50%<: +++

Table 2 lists the tested compounds including those that reduced the release of glycerol (+, ++, or +++). The lipolytic rate of muscle triglycerides is associated with metabolic dysfunction including insulin resistance and liver steatosis. Compounds that lower lipolysis of adipocytes may improve metabolic function including improving insulin sensitivity and reducing liver steatosis, thus improving outcomes in patients with diabetes mellitus (e.g., prediabetes or type II diabetes) obesity, and hyperlipidemia.

### Example 4. In Vivo Evaluation of Acylated Hydroxybenzoic Acids for Metabolic Disorders

Acylated active agents disclosed herein may be useful in the modulation of metabolic markers and for the treatment of metabolic disorders. Acylated active agents disclosed herein may also be useful in the modulation of NAFLD markers and for the treatment of NAFLD (e.g., NASH). This example demonstrates the capability of exemplary acylated active agents, compounds 3 and 4, to induce weight loss and improve metabolic markers (e.g., improve glucose tolerance) in a subject.

C57BL/6 mice were divided into nine cohorts, as listed in Table 3.

TABLE 3

| | Base Diet + | # of | | Dosing | |
|---|---|---|---|---|---|
| Model | Test Article* | animals | Dose** | Regimen | Route |
| LFD | LFD only | 12 | | | |
| HFD | HFD only | 12 | | | |
| HFD | HFD + | 12 | 6% of | Ad | Diet |
| | compound 1 | | compound 1 | libitum | |
| HFD | HFD + | 12 | 5% of | Ad | Diet |
| | compound 2 | | compound 2 | libitum | |
| HFD | HFD + | 12 | 7% of | Ad | Diet |
| | compound 3 | | compound 3 | libitum | |
| HFD | HFD + | 12 | 5% of | Ad | Diet |
| | compound 4 | | compound 4 | libitum | |
| HFD | HFD + | 12 | 6% of | Ad | Diet |
| | compound 5 | | compound 5 | libitum | |
| HFD | HFD + | 12 | | 3x | Subcu- |
| | Vehicle | | | weekly | taneously |
| HFD | HFD + | 12 | 246.8 μg/kg of | 3x | Subcu- |
| | Semaglutide | | semaglutide | weekly | taneously |

*In Table 3, HFD means high fat diet, and LFD means low fat diet, and vehicle is 4.9% DMSO in saline.
**In Table 3, dose percentages refer to weight percentage relative to the high fat diet.

Animals were allowed free access to food and drinking water for the entire study. Animals were weighed on a regular basis, and food and drinking water consumption monitored. Plasma and stool samples were collected at the beginning of the study and 42 days into the study. Additional blood was drawn from fasted mice 58 days into the study. Insulin tolerance tests were performed 72 or 73 days into the study. Following an approximate 4 h fast, 0.5 Units/kg insulin was administered intraperitoneally. Blood was collected pre-insulin challenge (t=0) and at t=15, 30, 60, 90, and 120 min following insulin challenge. Oral glucose tolerance tests were performed 79 or 80 days into the study. Following an approximate 4 h fast, 2 g/kg glucose was administered by oral gavage. Blood was collected pre-insulin challenge (t=0) and at t=15, 30, 60, 90, and 120 min following glucose challenge. At the end of the study, body weight, food consumption, blood glucose, serum ALT levels, serum AST levels, serum cholesterol (total cholesterol. HDL, and LDL levels), serum triglycerides levels, liver triglyceride levels, liver cholesterol levels, liver weight, subcutaneous were measured in all mice.

These samples and tests were used to measure disease makers.

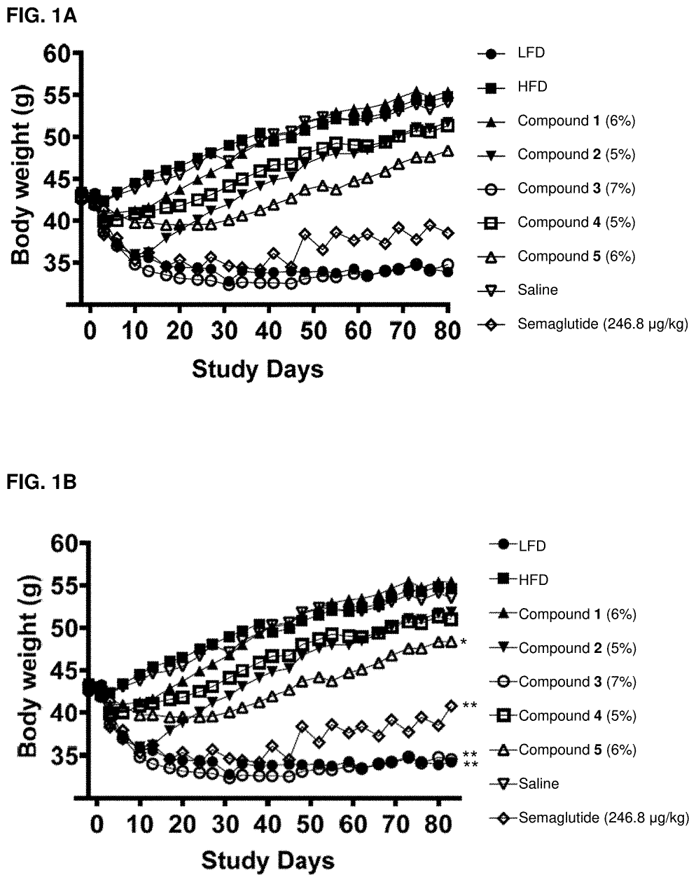

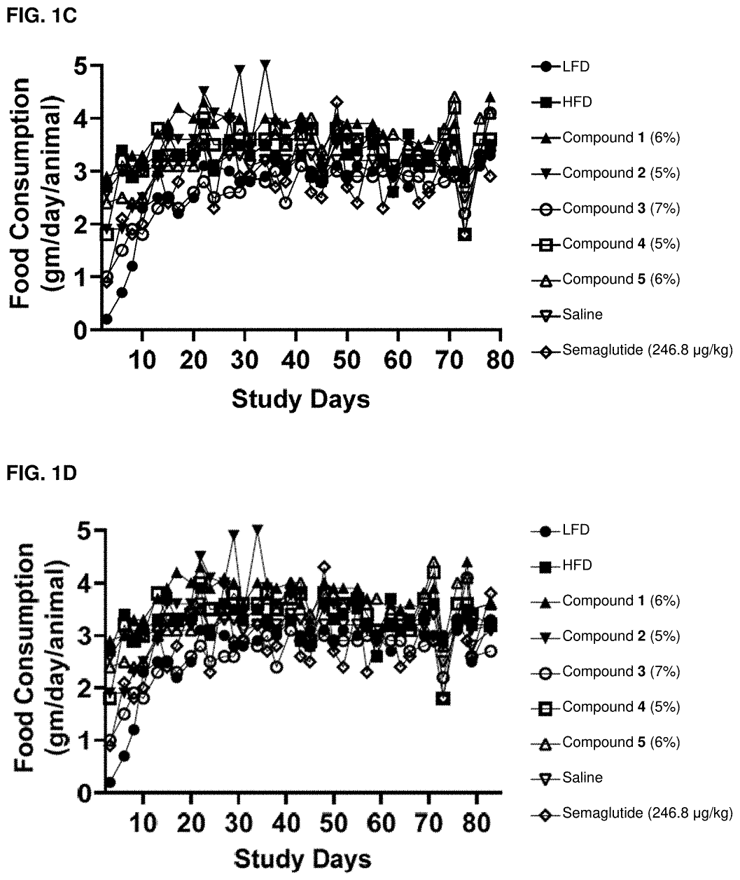

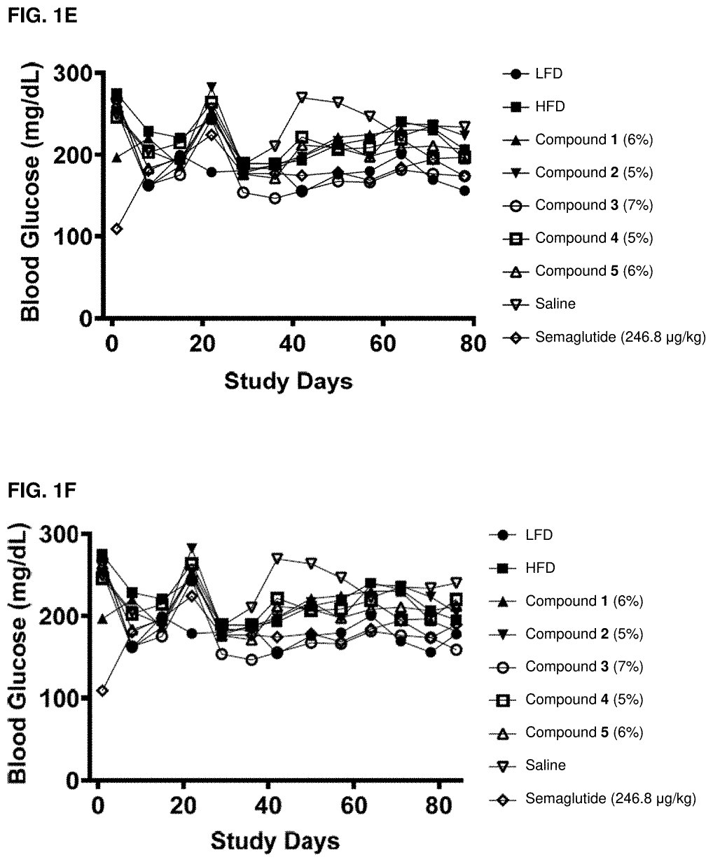

Results of this study are illustrated in FIGS. 1A, 1B, 1C, 1D, 1E, 1F, 2A, 2B, 3A, 3B, 4A, 4B, 4C, 4D, 4E, 5A, 5B, 6A, 6B, 6C, 6D, 7A, 7B, 7C, 8A, and 8B.

FIGS. 1A, 1B, 1C, 1D, 1E, and 1F show that animals in the HFD+compound 3 cohorts underwent weight loss despite being fed high-fat diets without significant appetite suppression. The body weights of animals in the HFD+compound 3 cohort were indistinguishable from those in the LFD cohort.

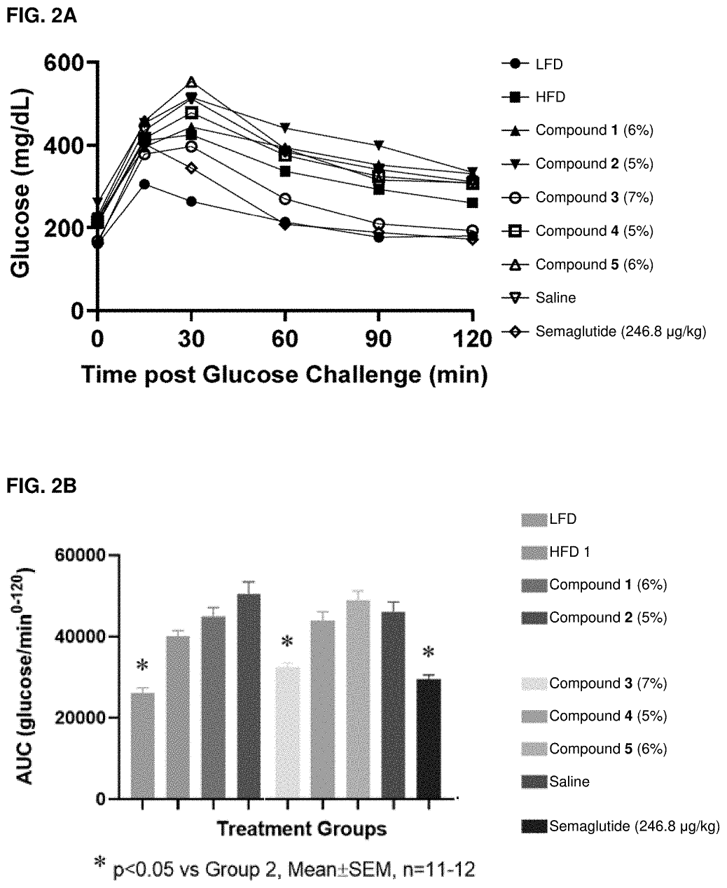

FIGS. 2A and 2B show that glucose tolerance of animals in the HFD+compound 3 cohorts exceed that of animals in the HFD only cohort.

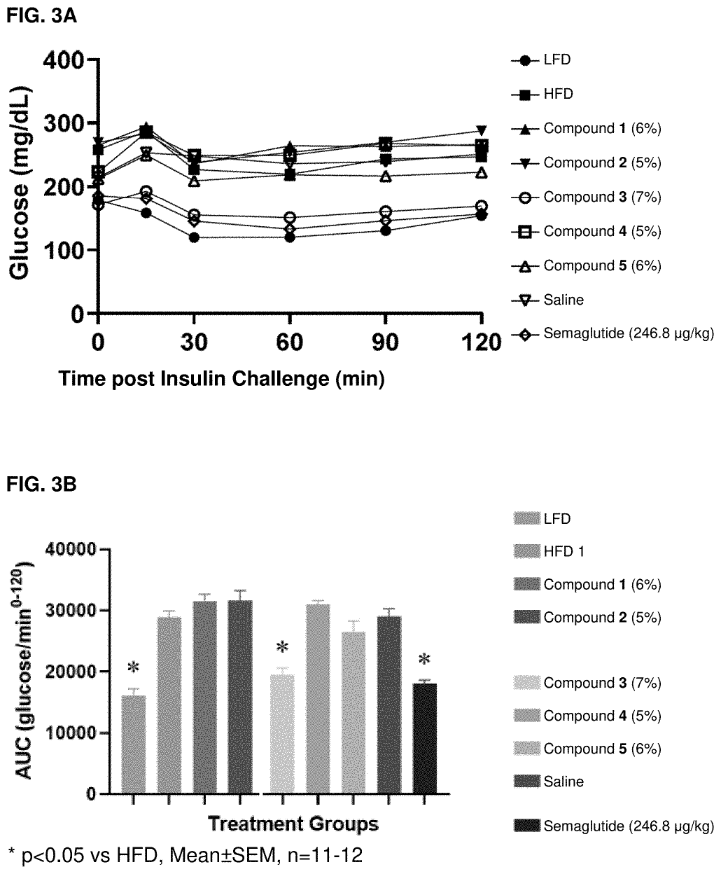

FIGS. 3A and 3B show that insulin tolerance of animals in the HFD+compound 3 cohorts exceed that of animals in the HFD only cohort.

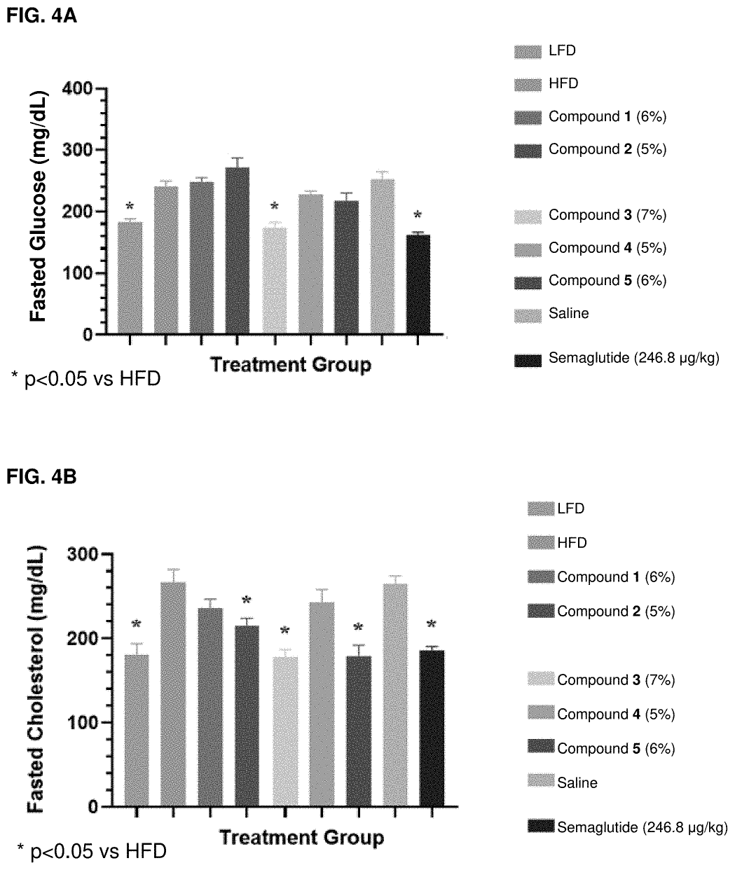

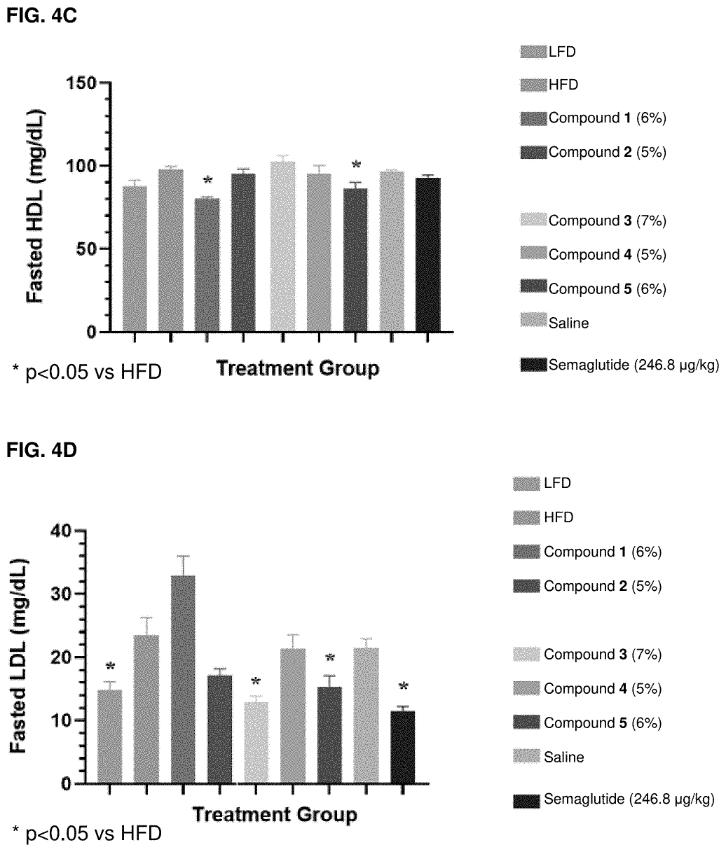

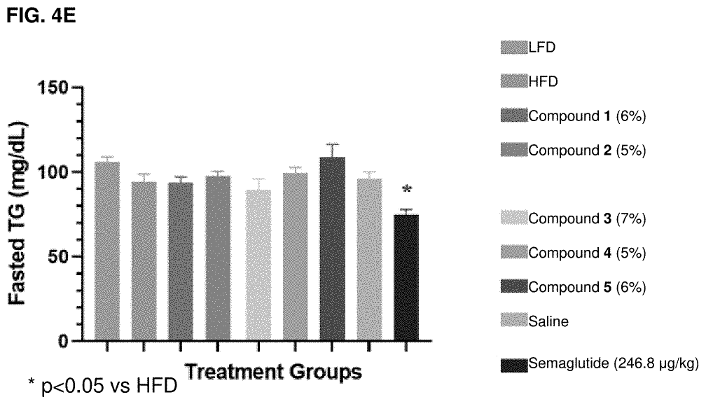

FIGS. 4A, 4B, 4C, 4D, and 4E show the fasted glucose levels, fasted cholesterol levels, fasted high density lipoprotein (HDL) levels, fasted low density lipoproptein (LDL) levels, and fasted triglyceride levels, respectively, in the tested animals at day 58 of the study. A single asterisk in these figures indicates an observation of a statistically significant reduction in the metabolic marker level in the test cohort relative to the HFD only cohort. In particular, FIGS. 4A, 4B, and 4D show that compounds of the invention can lower fasted glucose, cholesterol, and LDL levels when compared to the HFD only cohort.

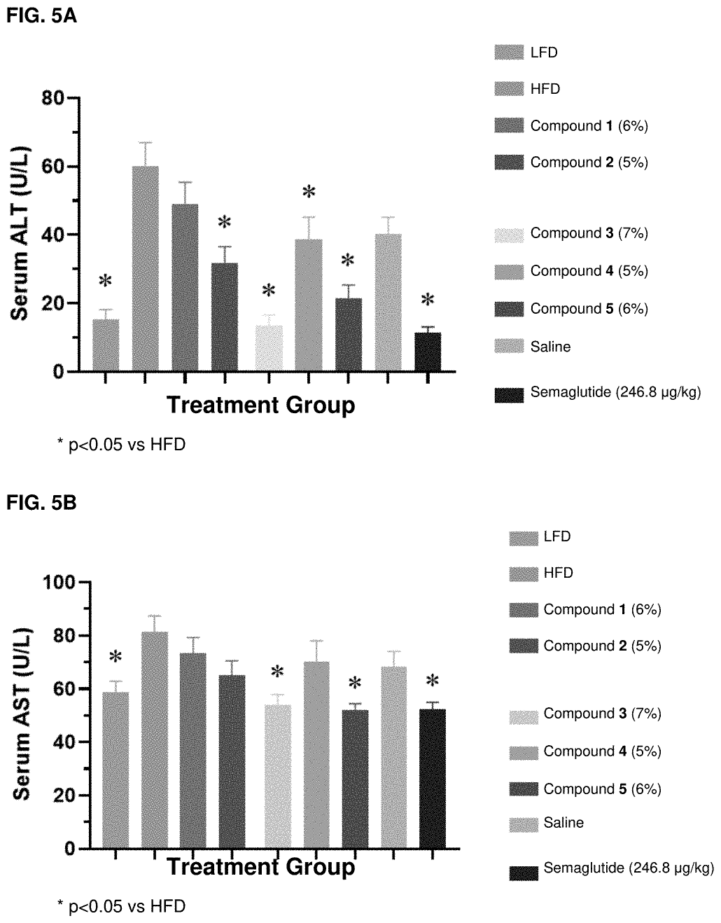

FIGS. 5A and 5B show serum ALT and AST levels in the tested animals at the end of the study. Alanine aminotransferase (ALT) and aspartate aminotransferase (AST) are liver enzymes. Elevated levels of ALT and AST are associated with medical conditions such as metabolic syndrome and liver injury. FIGS. 5A and 5B thus indicate that animals in the HFD+compound 3 cohort had significantly improved liver function relative to the animals in the HFD control group.

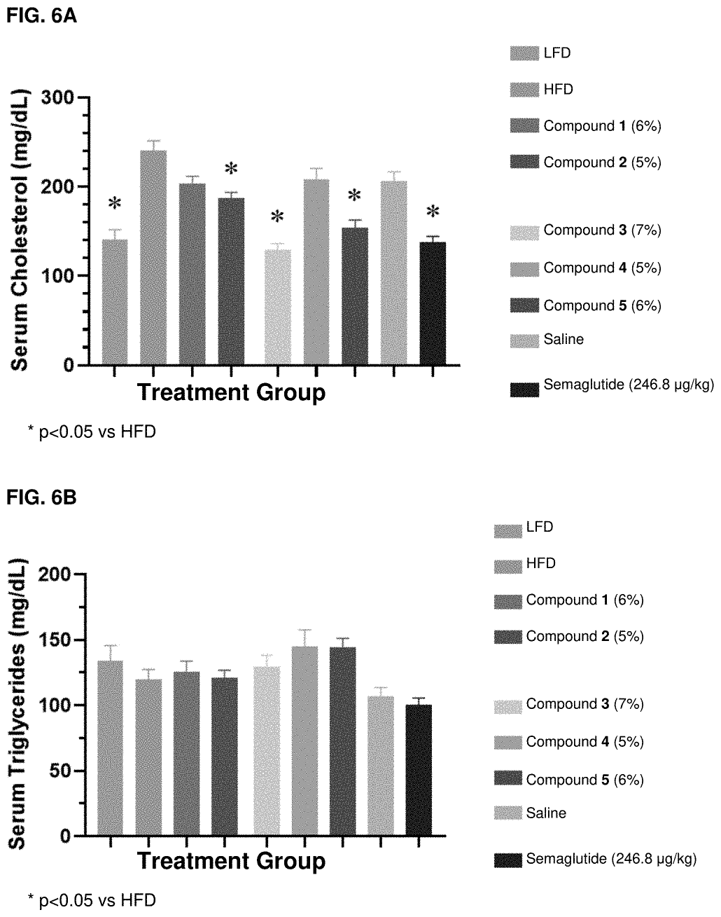

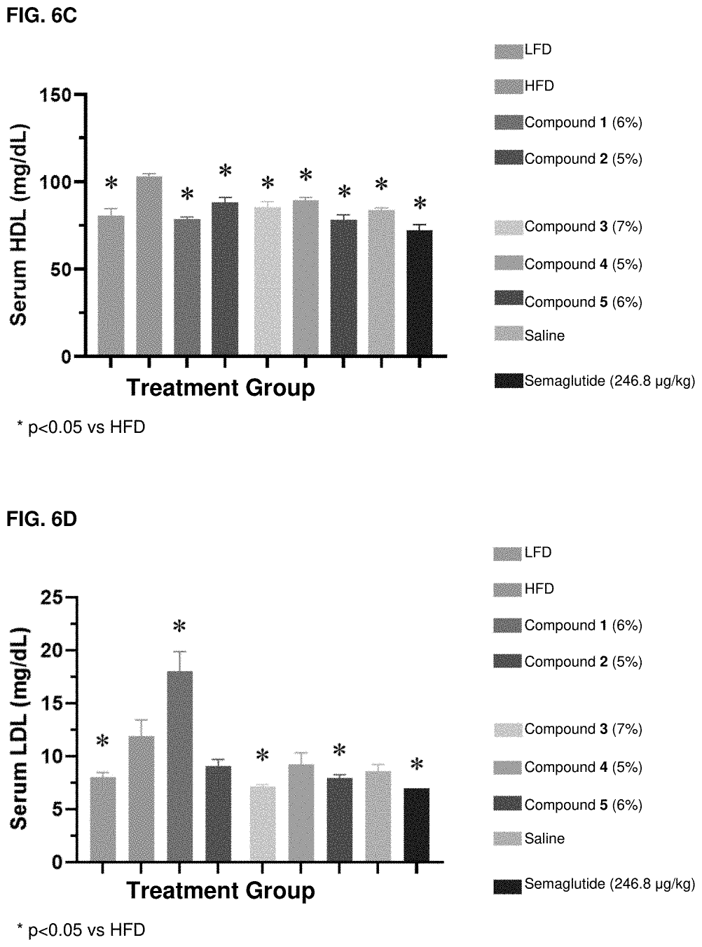

FIGS. 6A, 6B, 6C, and 6D demonstrate that total cholesterol levels were elevated in the animals in the HFD cohort relative to the animals in the LFD cohort. These FIGS. also show that animals in the HFD+compound 3 cohort had significantly improved total cholesterol and LDL levels at the end of the study relative to the animals in the HFD cohort.

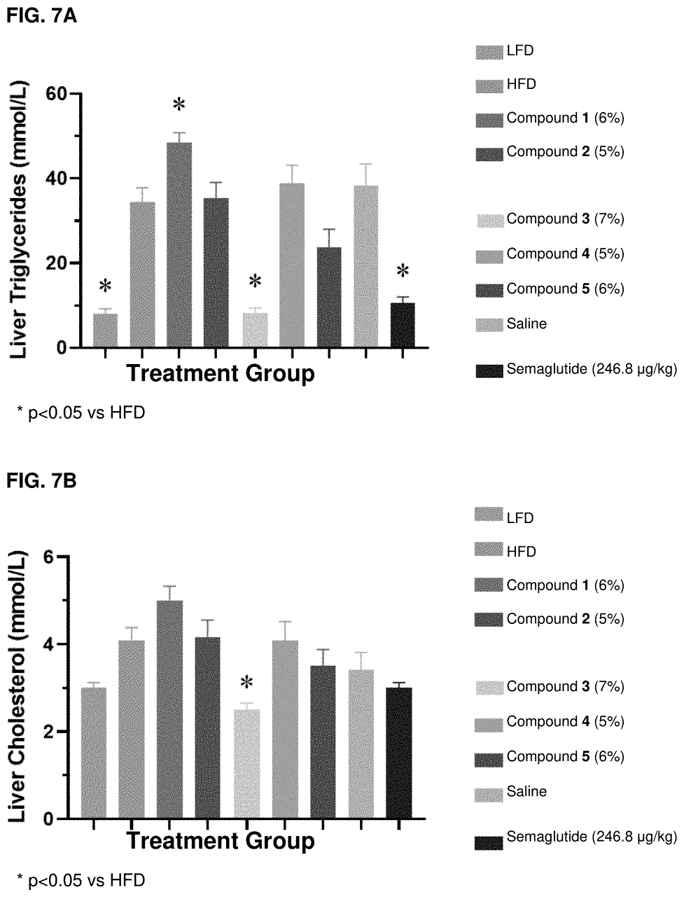

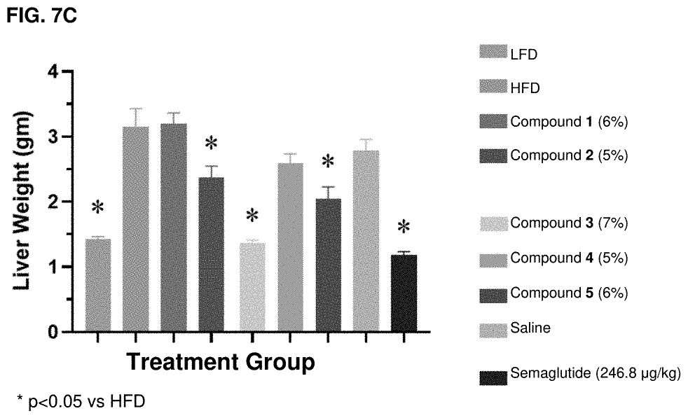

FIGS. 7A, 7B, and 7C provide characteristics of the livers from tested mice. Liver weight, triglyceride levels, and cholesterol levels are indicative of liver function and lipid metabolism

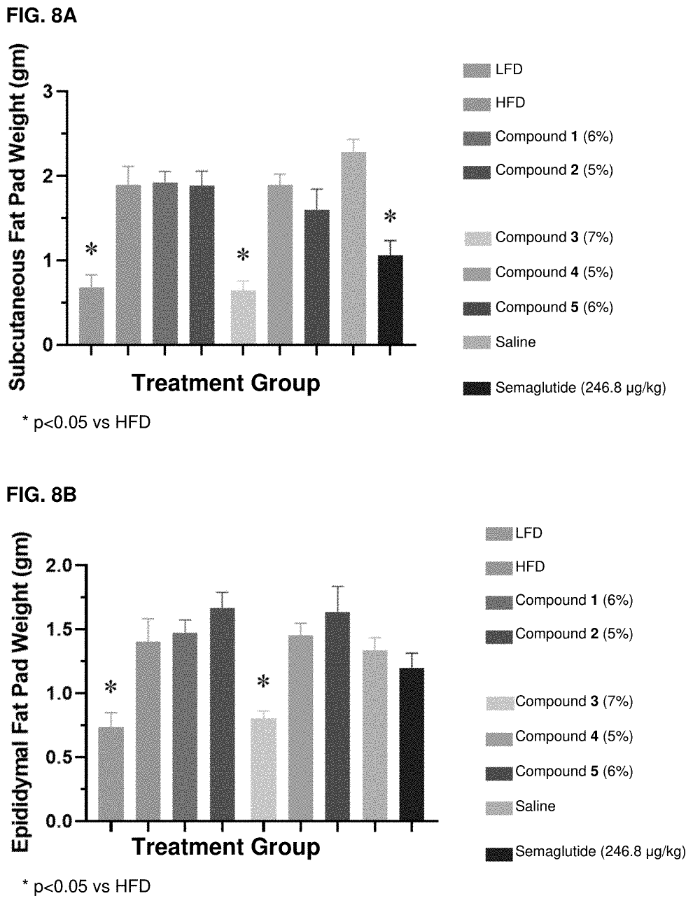

FIGS. 8A and 8B demonstrate that mice in the LFD and in the HFD+compound 3 cohorts had significantly less (p<0.05) fat accumulation than mice in the HFD control cohort.

The results of this study show that exemplary acylated active agents (e.g., acylated hydroxybenzoic acids, e.g., compound 3) can induce weight loss and improve metabolic markers (e.g., glucose tolerance, insulin tolerance, and cholesterol levels) in a subject.

### OTHER EMBODIMENTS

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Other embodiments are in the claims.

What is claimed is:

1. A unit dosage form comprising at least 0.5 g of a compound of formula (I): 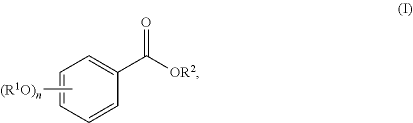
or a pharmaceutically acceptable salt thereof, wherein:
n is 1, 2, 3, 4, or 5;
each R¹ is independently H, alkyl, or acyl; and
R² is H or alkyl;
provided that the compound comprises at least one fatty acid acyl.

2. The unit dosage form of claim 1, wherein n is 2.

3. The unit dosage form of claim 1, wherein the compound is a compound of formula (IA): 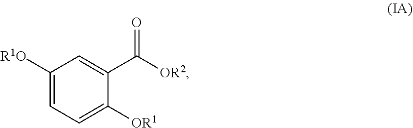
or a pharmaceutically acceptable salt thereof.

4. The unit dosage form of any one of claims 1 to 3, wherein each R¹ is independently acyl.

5. The unit dosage form of claim 4, wherein each R¹ is independently a short chain fatty acid acyl.

6. The unit dosage form of claim 1, wherein the compound is: 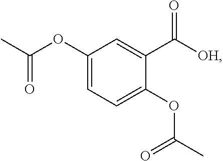 [SMILES: CC(=O)Oc1ccc(OC(C)=O)c(C(=O)O)c1]
or a pharmaceutically acceptable salt thereof.

7. The unit dosage form of any one of claims 1 to 6, wherein the unit dosage form comprises at least 1 g of the active agent.

8. The unit dosage form of any one of claims 1 to 6, wherein the unit dosage form comprises at least 2 g of the active agent.

9. The unit dosage form of any one of claims 1 to 8, wherein the unit dosage form comprises 10 g or less of the active agent.

10. The unit dosage form of any one of claims 1 to 8, wherein the unit dosage form comprises 9 g or less of the active agent.

11. The unit dosage form of any one of claims 1 to 8, wherein the unit dosage form comprises 8 g or less of the active agent.

12. The unit dosage form of any one of claims 1 to 8, wherein the unit dosage form comprises 7 g or less of the active agent.

13. The unit dosage form of any one of claims 1 to 8, wherein the unit dosage form comprises 6 g or less of the active agent.

14. The unit dosage form of any one of claims 1 to 8, wherein the unit dosage form comprises 5 g or less of the active agent.

15. The unit dosage form of any one of claims 1 to 14, wherein the unit dosage form is a food additive unit dosage form, a pharmaceutical unit dosage form, or a dietary supplement unit dosage form.

16. The unit dosage form of claim 15, wherein the unit dosage form is a food additive unit dosage form that is a serving of a food product.

17. The unit dosage form of claim 15, wherein the unit dosage form is a pharmaceutical unit dosage form.

18. The unit dosage form of claim 15, wherein the unit dosage form is a dietary supplement unit dosage form.

19. A method of modulating a metabolic marker or a nonalcoholic fatty liver disease marker, the method comprising administering an effective amount of an active agent to a subject in need thereof, wherein the active agent is an acylated hydroxybenzoic acid, a pharmaceutically acceptable salt thereof, or an ester thereof.

20. The method of claim 19, wherein the method is for modulating a metabolic marker.

21. The method of claim 19 or 20, wherein the metabolic marker is for an obesity disorder.

22. The method of claim 19 or 20, wherein the metabolic marker is for type II diabetes, prediabetes, insulin resistance, metabolic syndrome, hypercholesterolemia, or hyperlipidemia.

23. The method of claim 19, wherein the method is for modulating a nonalcoholic fatty liver disease marker.

24. A method of treating a metabolic disorder or nonalcoholic fatty liver disease, the method comprising administering an effective amount of an active agent to a subject in need thereof, wherein the active agent is an acylated hydroxybenzoic acid, or a pharmaceutically acceptable salt thereof, or an ester thereof.

25. The method of claim 24, wherein the method is for treating a metabolic disorder.

26. The method of claim 24 or 25, wherein the metabolic disorder is an obesity disorder.

27. The method of claim 24 or 25, wherein the metabolic disorder is type II diabetes, prediabetes, insulin resistance, metabolic syndrome, hypercholesterolemia, or hyperlipidemia.

28. The method of claim 24, wherein the method is for treating nonalcoholic fatty liver disease.

29. The method of claim 24 or 28, wherein the subject suffers from or is diagnosed with nonalcoholic steatohepatitis.

30. The method of claim 24, 28, or 29, wherein the method treats or reduces liver fibrosis.

31. A method of improving glucose or insulin tolerance, of reducing cholesterol levels, of reducing blood sugar levels, or of maintaining a healthy body weight in a subject in need thereof, the method comprising administering to the subject an effective amount of an active agent to a subject in need thereof, wherein the active agent is an acylated hydroxybenzoic acid, a pharmaceutically acceptable salt thereof, or an ester thereof.

32. The method of claim 31, wherein the method is of improving glucose tolerance.

33. The method of claim 31, wherein the method is of improving insulin tolerance.

34. The method of claim 31, wherein the method is of reducing blood sugar levels.

35. The method of claim 34, wherein the blood sugar levels are elevated prior to the administering step.

36. The method of claim 31, wherein the method is of reducing cholesterol levels.

37. The method of claim 36, wherein the cholesterol levels are total cholesterol levels.

38. The method of claim 36, wherein the cholesterol levels are serum LDL levels.

39. The method of any one of claims 31 to 38, wherein the subject is suffering from or is at risk of type II diabetes, prediabetes, insulin resistance, metabolic syndrome, or hypercholesterolemia.

40. The method of any one of claims 19 to 39, wherein total fat percentage, cellular adiposity, body mass index, rate of weight gain, abdominal fat quantity, ratio of white to brown fat, level of lipogenesis, or level of fat storage is reduced following the step of administering.

41. The method of any one of claims 19 to 39, wherein total fat percentage, cellular adiposity, body mass index, abdominal fat quantity, or ratio of white to brown fat is reduced following the step of administering.

42. The method of any one of claims 19 to 41, wherein the subject is overweight.

43. The method of any one of claims 19 to 41, wherein the subject suffers from obesity.

44. The method of any one of claims 19 to 41, wherein the subject suffers from severe obesity, morbid obesity, or super obesity.

45. The method of any one of claims 19 to 41, wherein the subject has a body mass index of at least 25 kg/m².

46. The method of any one of claims 19 to 41, wherein the subject has a body mass index of at least 28 kg/m².

47. The method of any one of claims 19 to 41, wherein the subject has a body mass index of at least 30 kg/m².

48. The method of any one of claims 19 to 41, wherein the subject has a body mass index of at least 35 kg/m².

49. The method of any one of claims 19 to 41, wherein the subject has a body mass index of at least 45 kg/m².

50. The method of any one of claims 19 to 49, wherein the level of insulin, GLP-1, or PYY is increased following the administration of the active agent to the subject.

51. The method of any one of claims 19 to 50, wherein the level of blood sugar or hemoglobin A1c is reduced following the administration of the active agent to the subject.

52. The method of any one of claims 19 to 51, wherein the glucose tolerance is increased following the administration of the active agent to the subject.

53. The method of any one of claims 19 to 52, wherein the method reduces the level of alanine transaminase in the blood of the subject by at least 1% relative to the level of alanine transaminase in the blood of the subject prior to the administering step.

54. The method of any one of claims 19 to 53, wherein the method reduces the level of aspartate transaminase in the blood of the subject by at least 1% relative to the level of aspartate transaminase in the blood of the subject prior to the administering step.

55. The method of any one of claims 19 to 54, wherein the method reduces the liver weight of the subject by at least 1% relative to the liver weight of the subject prior to the administering step.

56. The method of any one of claims 19 to 55, wherein the subject is a human.

57. The method of any one of claims 19 to 55, wherein the subject is a cat or dog.

58. The method of any one of claims 19 to 57, wherein the method comprises orally administering the active agent to the subject.

59. The method of claim 58, wherein, following oral administration to the subject, the active agent is cleavable in the gastrointestinal tract of the subject.

60. The method of any one of claims 19 to 59, wherein, upon cleavage, the active agent releases at least one fatty acid.

61. The method of claim 60, wherein the fatty acid is a short chain fatty acid.

62. The method of claim 61, wherein the short chain fatty acid is acetic acid, propionic acid, or butyric acid.

63. The method of claim 62, wherein the short chain fatty acid is acetic acid.

64. The method of claim any one of claims 19 to 63, wherein the active agent comprises gentisic acid.

65. The method of any one of claims 19 to 60, wherein the active agent is a compound of formula (I): 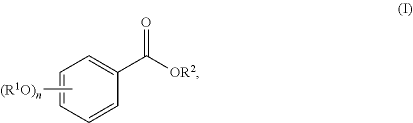
or a pharmaceutically acceptable salt thereof, wherein:
n is 1, 2, 3, 4, or 5;
each R¹ is independently H, alkyl, or acyl; and
R² is H or alkyl;
provided that the compound comprises at least one fatty acid acyl.

66. The method of claim 65, wherein n is 2.

67. The method of claim 65 or 66, wherein the compound is a compound of formula (IA): 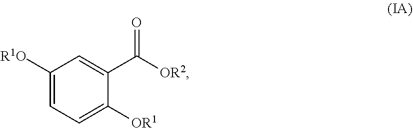
or a pharmaceutically acceptable salt thereof.

68. The method of any one of claims 65 to 67, wherein each R¹ is independently acyl.

69. The method of claim 68, wherein each R¹ is independently a short chain fatty acid acyl.

70. The method of claim 69, wherein the compound is: 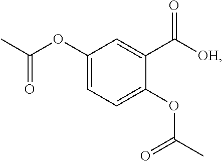 [SMILES: CC(=O)Oc1ccc(OC(C)=O)c(C(=O)O)c1]
or a pharmaceutically acceptable salt thereof.
